(19) United States
(12) Patent Application Publication
(10) Pub. No.: US 2011/0257641 A1
(43) Pub. Date: Oct. 20, 2011

(54) PHOTOTHERAPY FOR RENAL DENERVATION

(76) Inventors: Roger Hastings, Maple Grove, MN (US); Frank Ingle, Palo Alto, CA (US); Allan C. Shuros, St. Paul, MN (US); Eric Petersen, Maple Grove, MN (US); Mark Hollingsworth, Bloomington, MN (US)

(21) Appl. No.: 13/086,121

(22) Filed: Apr. 13, 2011

Related U.S. Application Data
(60) Provisional application No. 61/324,163, filed on Apr. 14, 2010.

(51) Int. Cl. A61B 18/24

(52) U.S. Cl. 606/15

(57) ABSTRACT

Apparatuses and methods facilitate delivery of optical or photoacoustic energy to innervated vascular that contributes to renal sympathetic nerve activity. The optical energy delivered may be of sufficient power to scan or image innervated renal or aortal tissue. The optical energy delivered may be of sufficient power to ablate innervated renal or aortal tissue, such as by thermal laser ablation or photoacoustic laser ablation. A catheter for intravascular or extravascular deployment supports an optical fiber arrangement comprising a coupling for receiving light from a laser light source. An optics arrangement is supported by the catheter and coupled to the optical fiber arrangement. The optics arrangement includes one or more optical elements arranged to receive the laser light and direct optical energy to target innervated tissue or a water source from which a cavitation bubble may be created and launched for acoustically shocking the target innervated tissue.

### RELATED APPLICATIONS

This application claims the benefit of Provisional Patent Application Ser. No. 61/324,163 filed on Apr. 14, 2010, to which priority is claimed pursuant to 35 U.S.C. §119(e) and which is hereby incorporated herein by reference in its entirety.

### TECHNICAL FIELD

The present invention is related to systems and methods for improving cardiac and/or renal function through neuromodulation, including disruption and termination of renal sympathetic nerve activity.

### BACKGROUND

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the bodies “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

### SUMMARY

Devices, systems, and methods of the invention are directed to modifying renal sympathetic nerve activity using phototherapy. Devices, systems, and methods of the present invention are directed to scanning or imaging innervated tissues that contribute to renal sympathetic nerve activity using phototherapy.

Embodiments of the invention are directed to apparatuses and methods for facilitating delivery of optical energy to innervated vascular that contributes to renal sympathetic nerve activity. Embodiments of the invention are directed to apparatuses and methods for facilitating delivery of optical energy to innervated tissue of one or both of a patient's renal artery and abdominal aorta.

According to some embodiments, apparatuses for facilitating delivery of optical energy to a renal artery of a patient include a catheter configured for deployment relative to the renal artery, and an optical fiber arrangement supported by the catheter and comprising a coupling for receiving laser light from a laser light source. An optics arrangement is supported by the catheter and coupled to the optical fiber arrangement. The optics arrangement includes one or more optical elements arranged to receive the laser light and project optical energy to a desired depth within innervated tissue at or proximate an outer wall of the renal artery. The optical energy is of sufficient power to ablate innervated tissue at or proximate the outer wall of the renal artery.

The optical energy is preferably sufficient to ablate the innervated renal artery tissue with negligible injury to inner wall tissue of the renal artery. For example, the one or more optical elements may be configured to project the optical energy from the catheter to the innervated renal artery tissue in a circular pattern, the optical energy sufficient to ablate the innervated renal artery tissue with negligible injury to inner wall tissue of the renal artery. In another example, the one or more optical elements may be configured to project the optical energy from the catheter to the innervated renal artery tissue in a spiral, the optical energy sufficient to ablate the innervated renal artery tissue with negligible injury to inner wall tissue of the renal artery.

In some embodiments, the optical energy delivered by apparatuses of the invention is of sufficient power to scan or image innervated tissue of one or both of the renal artery and abdominal aorta. In other embodiments, the optical energy delivered by apparatuses of the invention is of sufficient power to ablate innervated tissue of one or both of the renal artery and abdominal aorta. In further embodiments, apparatuses of the invention provide for delivery of relatively low optical energy to scan or image innervated tissue of one or both of the renal artery and abdominal aorta, and for delivery of relatively high optical energy to ablate innervated tissue of one or both of the renal artery and abdominal aorta.

Embodiments of the invention are directed to apparatuses that include a catheter or other elongated member configured for intravascular, extravascular, transvascular, or intra-to-extra vascular deployment relative to the renal artery, and an optical fiber arrangement supported by the catheter or member and comprising a coupling for receiving laser light from a laser light source. An optics arrangement is supported by the catheter or member and coupled to the optical fiber arrangement. The optics arrangement includes one or more optical elements arranged to receive the laser light and direct optical energy to innervated tissue of the renal artery.

Other embodiments of the invention are directed to apparatuses that include a catheter of the type described in the preceding paragraph and a laser light source. In some embodiments, the laser light source comprises a continuous wave laser. In other embodiments, the laser light source comprises a pulse laser, such as a femptosecond laser or a picosecond laser, for example.

In some embodiments, a continuous wave laser and an optics arrangement are configured to direct optical energy to innervated tissue of the renal artery for effecting thermal ablation of the innervated tissue. In other embodiments, an ultrafast laser and an optics arrangement are configured to direct optical energy to innervated tissue of the renal artery for effecting non-thermal ablation of the innervated tissue. Thermal laser denervation of innervated renal tissue may be performed to create a spiral lesion to reduce the risk of stenosis. Non-thermal laser denervation of innervated renal tissue may be performed to create circular or spiral lesions without injuring intervening tissue or risk of stenosis.

In accordance with various embodiments, the laser light source comprises an ultrafast laser, and the optics arrangement is configured to project optical energy to innervated tissue of the renal artery at a predetermined depth and with a transverse length or diameter at the predetermined depth. The optics arrangement may be configured to direct ablative optical energy in a desired pattern to innervated tissue of the renal artery without injuring intervening tissue of the renal artery, such as by use of an axicon or a conical lens arrangement.

In some embodiments, laser and optics arrangements are configured to deliver optical energy that ablates innervated tissue primarily by a photothermal mode of ablation. In other embodiments, laser and optics arrangements are configured to deliver optical energy that ablates innervated tissue primarily by an electromechanical mode of ablation. In various embodiments, for example, laser and optics arrangements are configured to deliver optical energy sufficient to create a cavitation bubble in innervated renal artery tissue at a predetermined depth, the cavitation bubble creating a rupture in the innervated renal artery tissue upon bursting.

In further embodiments, a cavitation bubble is formed by depositing sufficient optical energy at a site within the media or adventitia of an innervated target artery, and additional optical energy is deposited to preferentially grow the bubble (e.g., to a preferred size, in a preferred direction of growth, or launch in a preferred direction). For example, a cavitation bubble can be grown in the smooth muscle of the media so that bubble growth is directed preferentially radially outward and circumferentially as dictated by the arrangement of fibers of the smooth muscle. Depositing additional optical energy at the site causes the cavitation bubble to burst, thereby generating an acoustic shock wave which ruptures the innervated target tissue.

In other embodiments, an optical fiber arrangement comprises two optical fibers each coupling light from a laser light source to an optics arrangement. The optics arrangement is configured to project light emitted from the two optical fibers having sufficient energy to create a cavitation bubble at each of two spaced-apart sites in the innervated renal artery tissue at a predetermined depth, the cavitation bubbles merging to create a rupture in the innervated renal artery tissue upon bursting. The two optical fibers may be arranged in a co-parallel relationship or a non-parallel, angled relationship.

According to some embodiments, an apparatus for facilitating delivery of optical energy to a renal artery of a patient includes a catheter configured for intravascular deployment within a lumen of the renal artery, and an optical fiber arrangement supported by the catheter and comprising a coupling for receiving laser light from a laser light source. An optics arrangement is supported by the catheter and coupled to the optical fiber arrangement. The optics arrangement comprises one or more optical elements arranged to receive the laser light. A balloon arrangement is dimensioned for deployment within the lumen of the renal artery and comprises a fluid vessel containing at least water.

The balloon arrangement encompasses at least a portion of the optical fiber arrangement and the optics arrangement. The optical fiber and optics arrangements are configured to direct optical energy to the fluid vessel sufficient to create a cavitation bubble therein, the fluid vessel serving to direct an acoustic shock wave generated by bursting of the cavitation bubble to innervated target tissue of the renal artery. A surface of the fluid vessel and/or other portions of the balloon arrangement may comprise an acoustic reflector.

According to various embodiments, optics arrangements of the invention may include a mirror and at least one lens. The mirror redirects light propagated along the optical fiber arrangement through the at least one lens and out of the catheter. The mirror may be configured for rotation within the catheter in response to movement of a manual or motorized rotation mechanism coupled to the mirror.

In some embodiments, the fiber optic arrangement may include an array of optical fibers, and the optics arrangement may include one or more mirrors and at least one lens. The one or more mirrors redirect light propagated along the optical fiber array through the at least one lens and into the innervated renal artery tissue at a predetermined depth. The redirected light results in formation of a spot or spherical image formed in the innervated renal artery tissue at the predetermined depth. The optical fibers of the array may be offset from one another. In other embodiments, a microlens is disposed between an optical fiber array and one or more mirrors, wherein the one or more mirrors define a diffraction grating and the optical fibers of the array are in alignment with respect to one another.

According to various embodiments, an optics arrangement comprises one or more notches provided in an optical fiber of the optical fiber arrangement. The one or more notches extend from an outer surface of the optical fiber through fiber cladding and into core material of the optical fiber. The notches define a reflective surface inclined at a predetermined angle relative to a plane normal to a longitudinal axis of the optical fiber. The reflective surface acts as a reflection mirror such that a portion of light propagated through the optical fiber and impinging upon the reflective surface is reflected through an opposing wall of the optical fiber.

Other embodiments of the invention include a balloon arrangement dimensioned for deployment within the lumen of the renal artery. The balloon arrangement is configured to support at least a portion of the optical fiber arrangement and the optics arrangement at a relatively fixed location within the renal artery lumen when the balloon arrangement is expanded in its deployed configuration. The balloon arrangement may include a cryoballoon. The balloon arrangement may include a spiral guide rail over which the catheter traverses. The optics arrangement within the balloon arrangement may be situated at a location axially offset with respect to a longitudinal axis of a shaft of the balloon arrangement. For example, the optics arrangement may be situated normal to the longitudinal axis of the shaft of the balloon arrangement.

In accordance with various embodiments, a phototherapy system includes an optical coherence tomography (OCT) machine, a laser light source, an optical fiber coupler, and a catheter comprising one or more optical fibers. In a first mode, the OCT machine is coupled to the catheter via the optical fiber coupler for imaging innervated tissue of the renal artery or the abdominal aorta, and locating target tissue of the innervated tissue. In a second mode, the laser light source is coupled to the catheter via the optical fiber coupler for ablating the target tissue located by the OCT machine.

In further embodiments, an apparatus for facilitating delivery of optical energy to a renal artery of a patient includes a catheter configured for intravascular deployment within a lumen of the renal artery, and a phototherapy unit provided at a distal end of the catheter. The phototherapy unit comprises a light source configured to generate white light of an intensity sufficient to ablate innervated tissue of the renal artery. A balloon arrangement is dimensioned for deployment within the lumen of the renal artery and encompasses at least a portion of the phototherapy unit that comprises the white light source. The balloon arrangement comprises a reflector arrangement disposed on a region of the balloon proximate the phototherapy unit. The reflector arrangement serves to direct white light generated by the light source to innervated target tissue of the renal artery. A thermal transfer arrangement, which may be integral to the balloon or a separate arrangement, is configured to provide cooling to renal artery tissue adjacent the balloon.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3A illustrates various tissue layers of the wall of the renal artery;

FIGS. 3B and 3C illustrate a portion of a renal nerve;

FIG. 4 illustrates a phototherapy unit deployed in a renal artery and an external imaging system or device in accordance with embodiments of the invention;

FIG. 5 illustrates a phototherapy unit deployed in a renal artery which incorporates imaging and phototherapy delivery capabilities in accordance with embodiments of the invention;

FIG. 6 illustrates a phototherapy unit deployed in a renal artery using a stabilization arrangement in accordance with embodiments of the invention;

FIG. 7 shows different beam profiles of laser light emitted by a phototherapy unit deployed in a renal artery in accordance with embodiments of the invention;

FIG. 8 illustrates a phototherapy unit and a balloon arrangement deployed in a renal artery in accordance with embodiments of the invention;

FIGS. 9A and 9B illustrate phototherapy units having single and distributed phototherapy elements in accordance with embodiments of the invention;

FIG. 10 shows a phototherapy apparatus which includes a laser light source that generates laser light having a desired wavelength and intensity in accordance with embodiments of the invention;

FIGS. 11 and 12 illustrate phototherapy arrangements that can be deployed to denervate renal vasculature in accordance with embodiments of the invention;

FIGS. 13 and 14 show embodiments of a phototherapy arrangement that incorporate a balloon arrangement in accordance with the invention;

FIGS. 15-17 and 18A-18D show phototherapy arrangements that can be incorporated at the distal end of a catheter in accordance with embodiments of the invention;

FIG. 19 illustrates an optics arrangement of a phototherapy unit provided at a distal end of a catheter for focusing laser light at target tissue in accordance with embodiments of the invention;

FIG. 20A is an exaggerated sectional view of a renal artery and a laser beam emitted from an optics arrangement of a phototherapy unit positioned within a lumen of the renal artery in accordance with embodiments of the invention;

FIG. 20B is an exaggerated sectional view of a renal artery and a laser beam emitted from an optics arrangement of a phototherapy unit positioned at an extravascular location relative to the renal artery in accordance with embodiments of the invention;

FIGS. 21A-21C and 22A-22B illustrate various embodiments of a bundle of optical fibers that can be used to supply laser light to a phototherapy unit in accordance with embodiments of the invention;

FIGS. 23 and 24 illustrate optical fiber manifolds according to embodiments of the invention;

FIGS. 25 and 26 illustrate phototherapy units that employ a multiplicity of optical fibers in accordance with embodiments of the invention;

FIG. 27 illustrates a phototherapy unit that employs a high intensity white light source for ablating innervated renal vasculature in accordance with embodiments of the invention;

FIG. 28 is a diagram of various laser light source components of a phototherapy system in accordance with embodiments of the invention;

FIG. 29 is a diagram of a system that includes an optical imaging system, an optical ablation system, and an optical coupling arrangement that facilitates quick and easy coupling and decoupling of a catheter or probe to and from the two systems in accordance with embodiments of the invention;

FIGS. 30 and 31 illustrate phototherapy units that employ a multiplicity of optical fibers in accordance with embodiments of the invention;

FIGS. 32 and 33 illustrate an apparatus for denervating a patient's renal artery using a photoacoustic ablation arrangement in accordance with embodiments of the invention;

FIG. 34 illustrates an apparatus for denervating a patient's renal artery using a photoacoustic ablation arrangement in accordance with other embodiments of the invention;

FIG. 35 illustrates an apparatus for facilitating guided delivery of a phototherapy catheter to innervated renal vasculature using an intra-to-extra vascular methodology in accordance with embodiments of the invention; and

FIG. 36 shows a hinge mechanism that can be built into a catheter or other elongated member to enhance access to the renal artery in accordance with embodiments of the invention.

While the invention is amenable to various modifications and alternative forms, specifics thereof have been shown by way of example in the drawings and will be described in detail. It is to be understood, however, that the intention is not to limit the invention to the particular embodiments described. On the contrary, the intention is to cover all modifications, equivalents, and alternatives falling within the scope of the invention as defined by the appended claims.

### DETAILED DESCRIPTION

In the following description, references are made to the accompanying drawings which illustrate various embodiments of the invention. It is to be understood that other embodiments may be utilized, and structural and functional changes may be made to these embodiments without departing from the scope of the present invention.

Embodiments of the invention are directed to systems, devices, and procedures for delivering phototherapy to innervated renal vasculature. Embodiments of the invention are directed to systems, devices, and procedures for denervating renal vasculature using phototherapy to disrupt target tissue so that renal sympathetic nerve activity is permanently terminated. Embodiments of the invention are directed to systems, devices, and procedures for scanning innervated renal vasculature to locate target tissue for denervation and to evaluate the efficacy of phototherapy delivered to the target tissue.

Representative embodiments of the invention described herein are generally directed to phototherapy involving laser light, it being understood that other forms of phototherapy may be employed. Target innervated renal vasculature preferably includes renal nerves, renal ganglia, aortal ganglia and other nerves and ganglia that contribute to renal sympathetic nerve activity. Although preferred embodiments of the invention provide for complete and permanent termination of renal sympathetic nerve activity, various embodiments may be implemented to provide for temporary (e.g., weeks or months) cessation of renal sympathetic nerve activity.

Embodiments of the invention are directed to depositing optical energy at target innervated tissue having characteristics sufficient to cause necrotic coagulation of the innervated tissue. In some embodiments, a laser apparatus is implemented for intravascular deployment, and a cooling arrangement is used to cool the inner vessel wall to protect the intima from thermal damage. In other embodiments, a laser apparatus is implemented for extravascular, transvascular, or intra-to-extra vascular deployment, in which a cooling arrangement is not required (or is optional) because the target innervated tissue resides at or proximate the exterior of the vessel wall.

Further embodiments of the invention are directed to apparatuses and methodologies that employ photoacoustic cavitation as a mechanism for denervating target vascular tissue. According to various embodiments, laser light is deposited at a focal site or zone containing a liquid or gel (preferably containing water) at energies exceeding a vaporization threshold. Rapid heating and vaporization of the liquid or gel produces a cavitation bubble formed around the focal site or zone. Depositing additional optical energy into the cavitation bubble can cause the bubble to grow and migrate in a preferred direction. Depositing sufficient optical energy into the cavitation bubble causes the bubble to implode or explode.

Implosion or explosion of the cavitation bubble results in the release of an appreciable amount of acoustic energy in the form of an acoustic shock wave. Propagation of the acoustic shock wave through the target innervated tissue mechanically ruptures sheaths of the nerve fibers included within target tissue, thereby substantially preventing regeneration of these nerve fibers. Advantageously, photoacoustic denervation using cavitation in accordance with embodiments of the invention can effectively denervate targeted renal and aorta tissue without causing thermal damage to surrounding or intervening tissue.

In some embodiments, an intravascular balloon catheter includes or receives a phototherapy unit from which laser light is extracted and directed to a focal site or zone within the balloon that contains a liquid or gel (preferably containing water). For example, a fluid container such as a pouch, bladder or channel is disposed along all or a portion of the balloon wall. Water or saline is contained in, or circulates through, the pouch, bladder or channel. Laser light is directed at a focal site or zone within the fluid container of sufficient energy to cause the production and subsequent implosion/explosion of a cavitation bubble at the focal site or zone. It is noted that multiple cavitation bubbles may be produced using a laser arrangement capable of directing light to multiple foci within the fluid container. Implosion/explosion of the cavitation bubble(s) results in generation of an acoustic shock wave that propagates through the balloon and into the vessel wall, resulting in fracturing of neural sheaths of nerve fibers included within target tissue.

In other embodiments, an intravascular balloon catheter includes or receives a phototherapy unit from which laser light is extracted and directed to a focal site or zone within the media or adventitia layers of the target innervated vessel. Laser light is directed at the focal site or zone within the vessel tissue of sufficient energy to cause the production and subsequent implosion/explosion of a cavitation bubble or bubbles at the focal site or zone. Implosion/explosion of the cavitation bubble(s) results in generation of an acoustic shock wave that propagates through the media and/or adventitia an impinges on the renal nerve or ganglion, resulting in fracturing of neural sheaths of nerve fibers included within target tissue. The balloon may provide for circulation of a cooling fluid to ensure that the intima is protected against thermal damage that may result from absorption of photons at or near the intima.

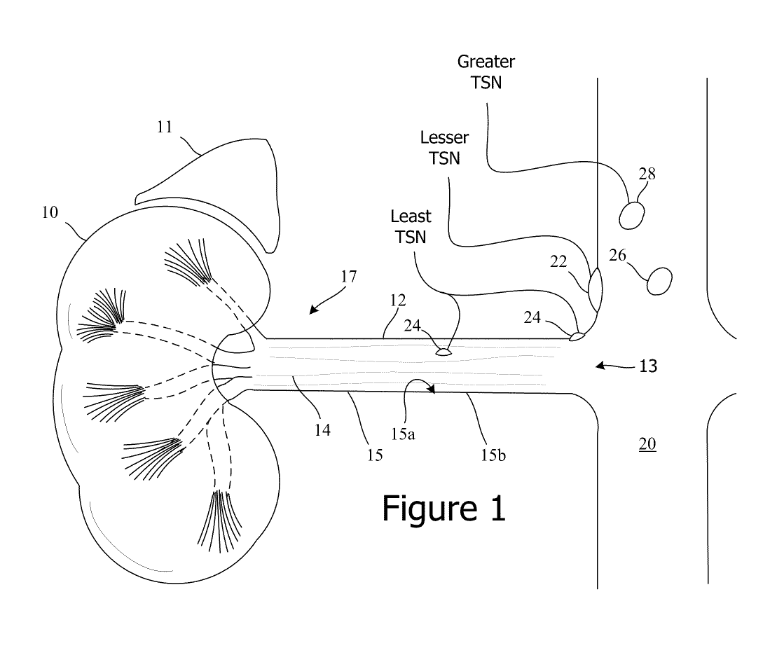

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the present invention. The renal artery 12 is purposefully shown to be disproportionately larger than the right kidney 10 and abdominal aorta 20 in order to facilitate discussion of various features and embodiments of the present disclosure.

The right and left kidneys are supplied with blood from the right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitrol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue.

The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

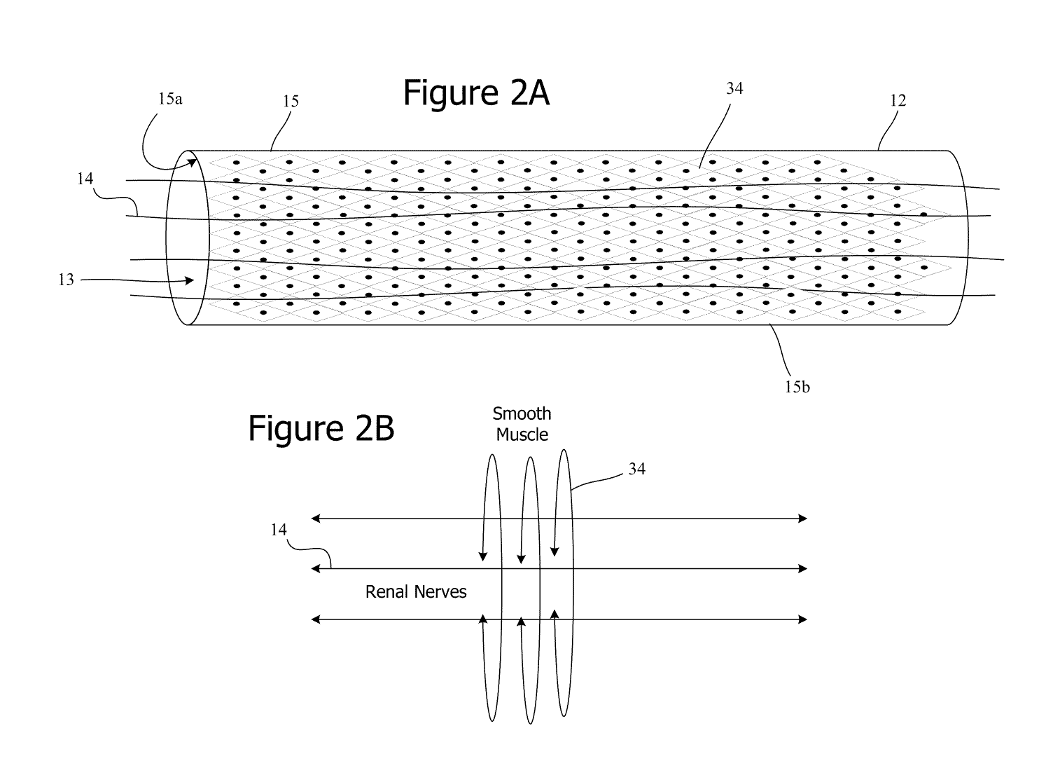

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

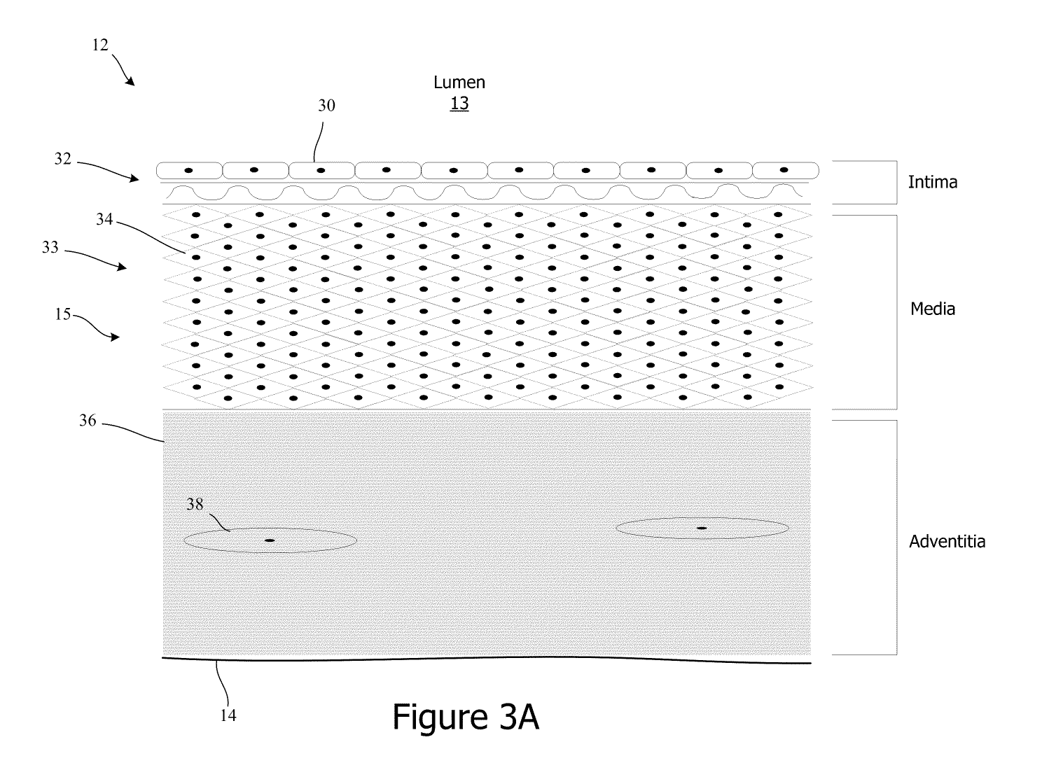

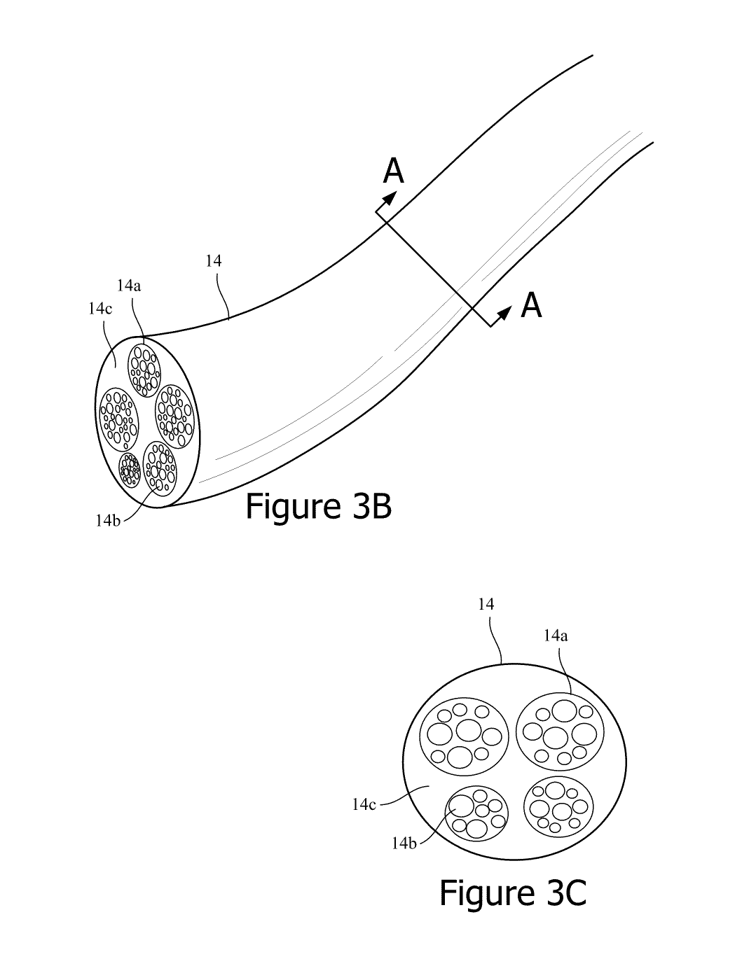

FIG. 3A shows a segment of a longitudinal cross-section through a renal artery, and illustrates various tissue layers of the wall 15 of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing.

A renal nerve 14 is shown proximate the adventitia 36 and extending longitudinally along the renal artery 12. The main trunk of the renal nerves 14 generally lies at or adjacent the adventitia of the renal artery 12, with certain branches coursing into the media to enervate the renal artery smooth muscle. For example, renal nerves may be situated in the adventitia proximate the outer wall of the renal artery (e.g., tunica adventitia) or within the vasa vasorum, such as the vasa vasorum externae.

In the context of the embodiments described herein, various terms are used for descriptive purposes that are well understood to those skilled in the art. Such terms are generally used by those skilled in the art in the context of ex vivo laser apparatuses or in vivo laser apparatuses that direct laser light into or through an aqueous transparent environment, such as the human eye. Embodiments of the invention are directed to laser apparatuses that are implemented for imaging and ablating vascular tissue. Laser light passing through vascular and other tissue of the body is subject to varying degrees of scattering and absorption.

In describing some embodiments of the invention, it is recognized that scattering and absorption of laser light passing through vasculature impacts imaging and/or ablation performance. Various parameters may be selected and/or adjusted to achieve a desired level of performance, including laser light wavelength, input polarization, energy, therapy duration, spot size, use of multiple beams of different wavelength, directing multiple beams from different directions, and use of local cooling, among others.

Appropriate selection of these and other parameters provide for “controlled scattering” of laser light propagating through innervated vasculature in accordance with embodiments of the invention. For example, controlled scattering can be achieved by using laser light for imaging or ablation in a preferred wavelength range (and perhaps from multiple directions) and polarization in order to facilitate absorption of a sufficient number of photons within target tissue located at typical target depths. Typical target depths have a range of about 1-4 mm, with a preferred range of about 0.5 mm-3 mm, and a more preferred range of about 0.5-2 mm.

In general terms, light interaction with biological tissue can be described using three parameters: a scattering coefficient (μₛ), an absorption coefficient (μₐ), and an anisotropy parameter (g) which describes the directional dependence of the scattered photons. The anisotropy parameter (g) describes the fraction of light forward scattered from an initial propagating direction s to s′. The reciprocal of the scattering (or absorption) coefficient is the average distance that a photon will travel before being scattered (or absorbed).

According to one approach, a user defined value of the anisotropy parameter (g) allows for the determination of the remaining two parameters for a specified wavelength range using known computational methods (e.g., an inverse adding-doubling method). In the case of strong forward scattering of biological tissues, such as vascular tissue, typical anisotropy values range from about 0.8 to about 0.95.

The scattering coefficient (μₛ), absorption coefficient (μₐ), and anisotropy parameter (g) may be obtained (or determined) for different tissues that are subject to scanning and ablating in accordance with embodiments of the invention. For example, these coefficients and parameters may be obtained or determined for various tissues of the renal artery, such as the intima, media, adventitia, and vasa vasorum. Fine tuning of laser imaging and ablation performance may be enhanced by knowledge of the scattering coefficient (μₛ), absorption coefficient (μₐ), and anisotropy parameter (g) for these tissues.

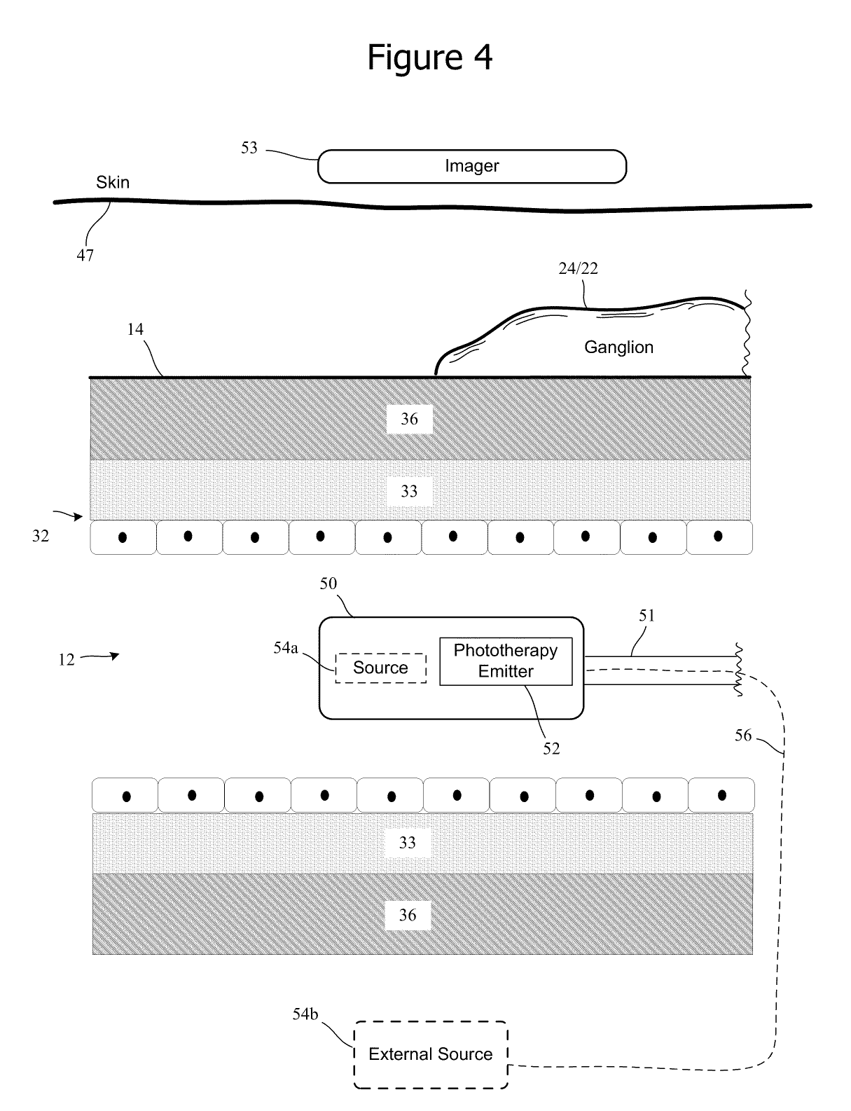

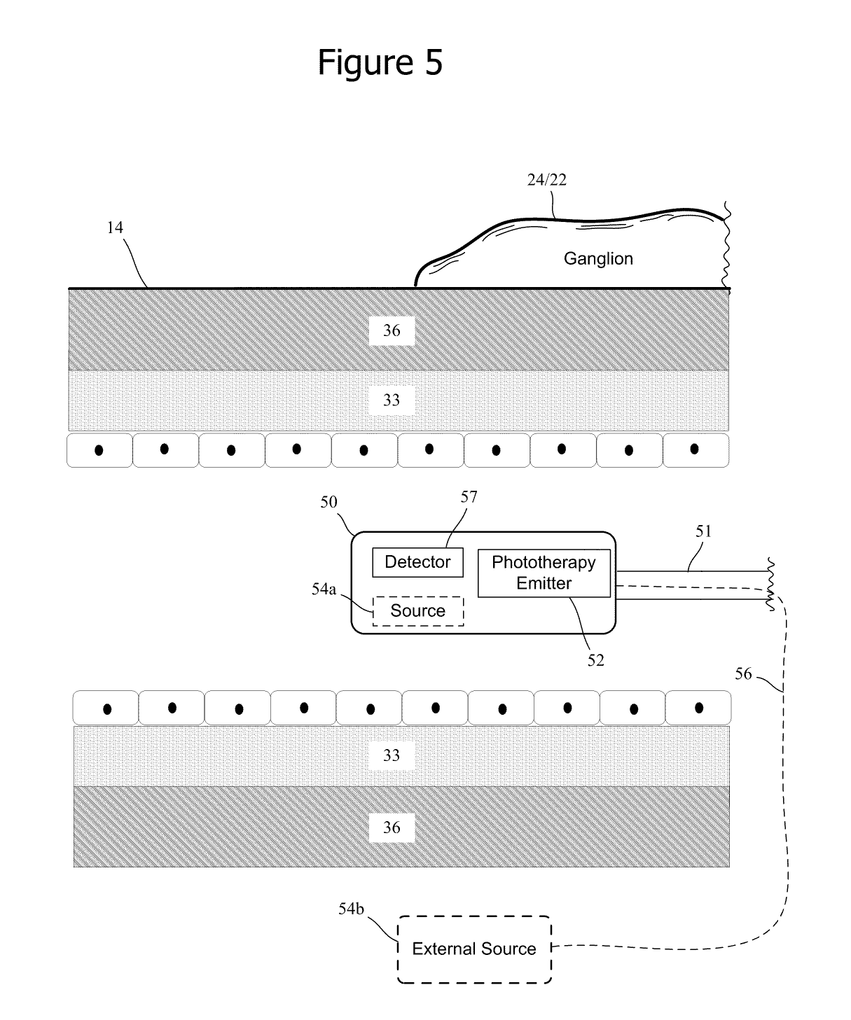

FIGS. 4 and 5 illustrate phototherapy treatment arrangements for denervating innervated renal and/or aortic tissue that contribute to renal sympathetic nerve activity in accordance with embodiments of the invention. FIGS. 4 and 5 show an exaggerated sectional view of a portion of a patient's renal artery 12. The tissue layers of the renal artery 12 shown in FIGS. 4 and 5 include the intima 32, which comprises the endothelium, the media 33, which includes smooth muscle, and the adventitia 36. A renal nerve 14 and a ganglion (e.g., renal ganglion 24 or aorticorenal ganglion 22) are shown on or proximate an outer section of the adventitia 36 for illustrative purposes.

A phototherapy unit 50 includes a phototherapy emitter 52 disposed in a housing to which a distal end of a catheter 51 is connected. The emitter 52 is coupled to a light source 54. The emitter 52 typically includes an optics arrangement to facilitate extraction of laser light received from the light source 54 and to direct laser light to target renal tissue. In some embodiments, as shown in FIG. 4, the phototherapy unit 50 includes a phototherapy emitter 52 and a separate imaging system 53 or device for imaging renal tissue and positioning the phototherapy unit 50 within the renal artery 12. The imaging system or device 53 may be external to the patient (i.e., outside the skin 47) or at least partially implantable, such as an endovascular imaging device (e.g., IVUS or intravascular ultrasound device). Suitable intravascular, extravascular, and extracorporeal apparatuses include magnetic resonance imaging (MRI), optical coherence tomography, and ultrasound apparatuses, for example.

In other embodiments, as shown in FIG. 5, the phototherapy unit 50 includes a phototherapy emitter 52 and a detector 57 or other local imaging device for imaging renal tissue and positioning the phototherapy unit 50 within the renal artery 12. In some embodiments, the detector 57 comprises a photodetector that receives light backreflected from the target tissue. Data associated with the backreflected light is communicated to an external system which produces imaging data and visual information useful for positioning the phototherapy unit 50 and evaluating the efficacy of a phototherapy procedure. In some embodiments, the detector 57 includes an ultrasonic transducer or other imaging device to facilitate imaging of the renal artery. In general, suitable locating apparatuses provide target depth or target range data that are used by the system computer to adjust one or more focus parameters of the emitter 52.

In FIGS. 4 and 5, the emitter 52 is coupled to an external light source 54b via a coupling 56. The external light source 54b is situated external to the renal artery, such as at a location outside the body (e.g., an external laser source). The coupling 56 is typically an optical coupling, such as an optical fiber or fiber bundle, which enters the renal vasculature at a suitable access vessel location (e.g., superior or inferior abdominal aorta). In other embodiments, the light source 54a is disposed within the housing of the phototherapy unit 50 and may draw power from a power source internal to the phototherapy unit 50 (battery, capacitor, energy harvesting device) or from a patient-external power source. The light source 54a may also be housed in a separate unit inside the body (e.g., a subcutaneous pocket or within the abdominal cavity, among other locations) and draw power from an internal power source or an external power source (e.g., via electromagnetic induction using an RF source external of the patient).

In various embodiments, the emitter 52 comprises a phototherapy apparatus capable of transmitting optical energy into the renal artery wall sufficient to disrupt target tissue that includes one or both of renal nerves 14 and ganglia 24/22. The optical energy transmitted by the emitter 52 is preferably sufficient to disrupt the target tissue so that renal sympathetic nerve activity is permanently terminated.

In other embodiments, the emitter 52 comprises a phototherapy apparatus capable of transmitting optical energy into the renal artery wall sufficient to facilitate locating of renal nerves 14 and ganglia 24/22 but insufficient to significantly disrupt renal sympathetic nerve activity (e.g., insufficient to effect permanent cessation of renal sympathetic nerve activity). In such embodiments, the emitter 52 may be used in combination with a detector 57 to facilitate imaging of renal artery and aortic tissue.

In further embodiments, the emitter 52 comprises a phototherapy apparatus or apparatuses capable of transmitting optical energy into the renal artery wall sufficient to facilitate locating of renal nerves 14 and ganglia 24/22 within target tissue and transmitting optical energy into the target tissue sufficient to significantly disrupt renal sympathetic nerve activity, such as by permanently terminating renal sympathetic nerve activity.

Locating target tissue may involve locating renal or aortic ganglia and/or artery tissue which includes renal nerves 14, such as the adventitia proximate the outer wall of the renal artery or the vasa vasorum externae. For example, one or more locating components of the phototherapy unit 50 may be used to scan the renal artery 12 or adjacent tissue that includes renal nerves and/or renal/aortal ganglia. The phototherapy unit 50 (or other locating apparatus, internal or external) may be controlled to scan for target tissue in deep layers of the adventitia and/or the vasa vasorum, such as the vasa vasorum externae which penetrates the outer adventitia (tunica adventitia).

In some embodiments, the phototherapy unit 50 is configured to selectively operate in a scan mode and a denervation mode, allowing the phototherapy unit 50 to locate target tissue in the scan mode and then permanently disrupt renal nerve fibers and ganglia within the target tissue in the denervation mode. Details of components and functionality that can be adapted for use in or by the phototherapy unit 50 are disclosed in U.S. Pat. Nos. 5,344,395 and 5,601,526, which are incorporated herein by reference.

In various embodiments, a single transducer operates as the emitter 52 and the detector 57. In other embodiments, one transducer operates as the emitter 52 and another transducer operates as the detector 57. In further embodiments, the transducer that is configured to delivery denervation therapy is also operative as a scanning transducer. In some embodiments, separate denervation and scanning transducers are employed. It is understood that the emitter and/or receiver components shown in the figures may define single transducer elements or an array of transducer elements.

Representative phototherapy apparatuses suitable for use in the emitter 52 include devices capable of delivering focused optical energy to target tissue that causes an increase in the temperature of the target tissue to a level that disrupts the target tissue and prevents chronic recovery of nerve fibers/ganglion in the target tissue resulting from the burn injury. Representative phototherapy apparatuses suitable for use in the emitter 52 include devices capable of delivering focused energy to target tissue that causes mechanical disruption of target tissue and prevents chronic recovery of nerve fibers/ganglion in the target tissue resulting from the mechanical disruption (e.g., cavitation microbubbles). Preferred phototherapy apparatuses for disrupting target tissue include those that achieve a desired level of disruption of target tissue while leaving adjacent or intervening tissue uninjured or negligibly injured (e.g., subject to healing without permanent adverse effects).

In accordance with various embodiments, the phototherapy unit 50 is configured so that it can be rotated and/or translated longitudinally within the lumen of the renal artery 12 to create a generally spiral ablation at a target depth in the renal artery wall. The spiral lesion may either be continuous or a sequential and overlapping line of ablated spots. In some embodiments, it may be desirable to ablate renal nerves 14 as they pass along the length of the renal artery 12, but it may not be desirable to ablate the renal artery in a circular fashion because of the risk of stenosis of the artery. Embodiments of a phototherapy unit 50 of the invention contemplate ablating a spiral shape in the renal artery which circles the artery at least once as the burned area spirals and translates along the artery wall.

In other embodiments, it may be desirable to ablate renal nerves 14 in a circular fashion without incurring undue risk of stenosis of the artery. Embodiments of a phototherapy unit 50 of the invention contemplate ablating a circumferential shape in the renal artery which circles the artery at least once. According to these embodiments, a phototherapy unit 50 of the invention includes an optics arrangement that projects optical energy deep into renal artery tissue (and beyond if needed, e.g., into the vasa vasorum), while leaving tissue adjacent or proximate the inner wall of the renal artery (e.g., the intima and at least a portion of the media) relatively unaffected or at most negligibly damaged.

One advantage of creating a circular or cylindrical lesion in the renal artery wall is that the longitudinal extent of the lesion is limited, which allows for repeated denervation procedures to be performed at untreated regions of the renal artery without undue risk of artery stenosis. For example, a circular or cylindrical lesion may be created near the ostium of the renal artery, leaving the majority of renal artery tissue untreated. Should additional renal denervation be required, a subsequent circular or cylindrical lesion may be created near the center or distal end of the renal artery. A mapping of renal artery lesion locations for a given patient may be stored to aid in avoiding previously treated regions of the artery when performing a subsequent ablation procedure.

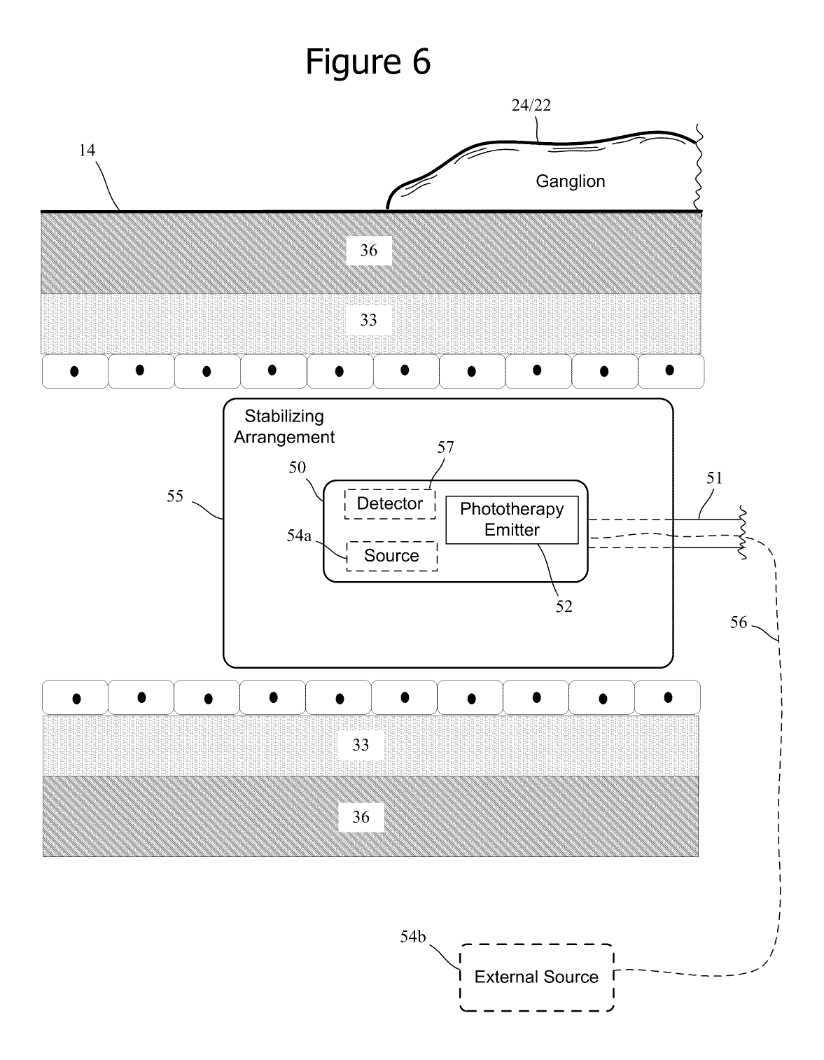

FIG. 6 shows another embodiment of a phototherapy treatment arrangement for denervating renal vasculature that contributes to renal sympathetic nerve activity in accordance with the invention. A support or stabilizing arrangement 55 is provided to aid in maintaining the phototherapy unit 50 at a relatively constant distance from the artery wall as the phototherapy unit 50 is translated and rotated within the lumen of the renal artery 12.

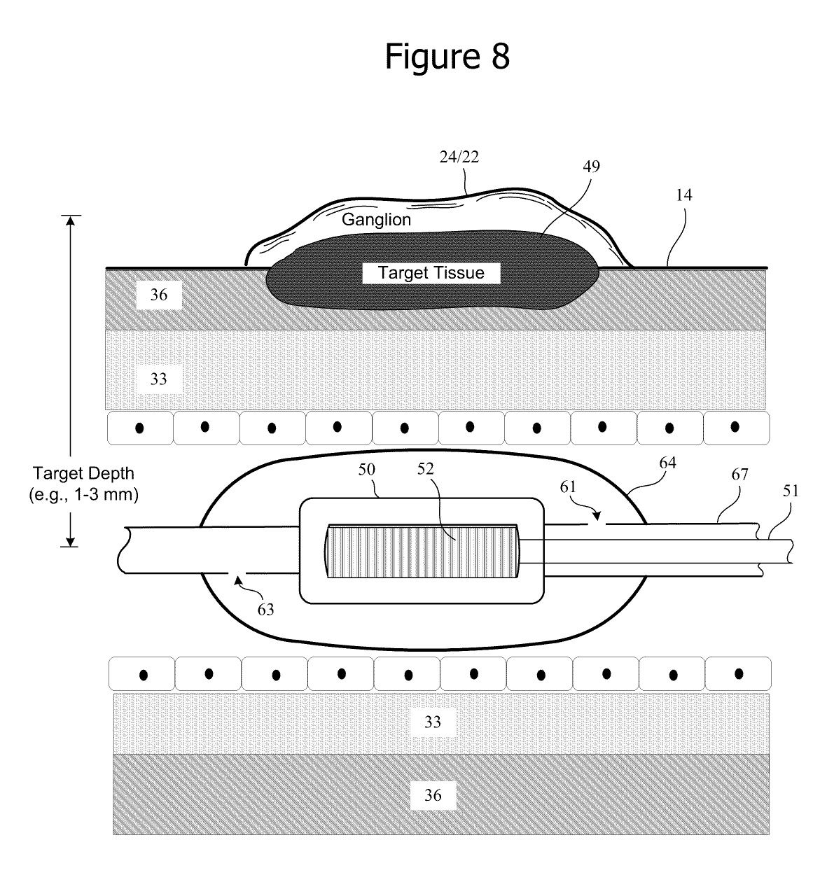

In various configurations, it is desirable to stabilize the position of the phototherapy unit 50 within the renal artery 12 so that the intensity of the optical energy emitted by the phototherapy unit 50 does not vary significantly with location, which could otherwise result in over-treated and under-treated regions. One approach to keeping the phototherapy unit 50 at a constant distance from the wall of the renal artery is to incorporate the into a balloon which can be expanded until it fills the arterial lumen, embodiments of which are discussed below with reference to FIGS. 8, 13, and 14. Other stabilizing arrangements 55 are contemplated.

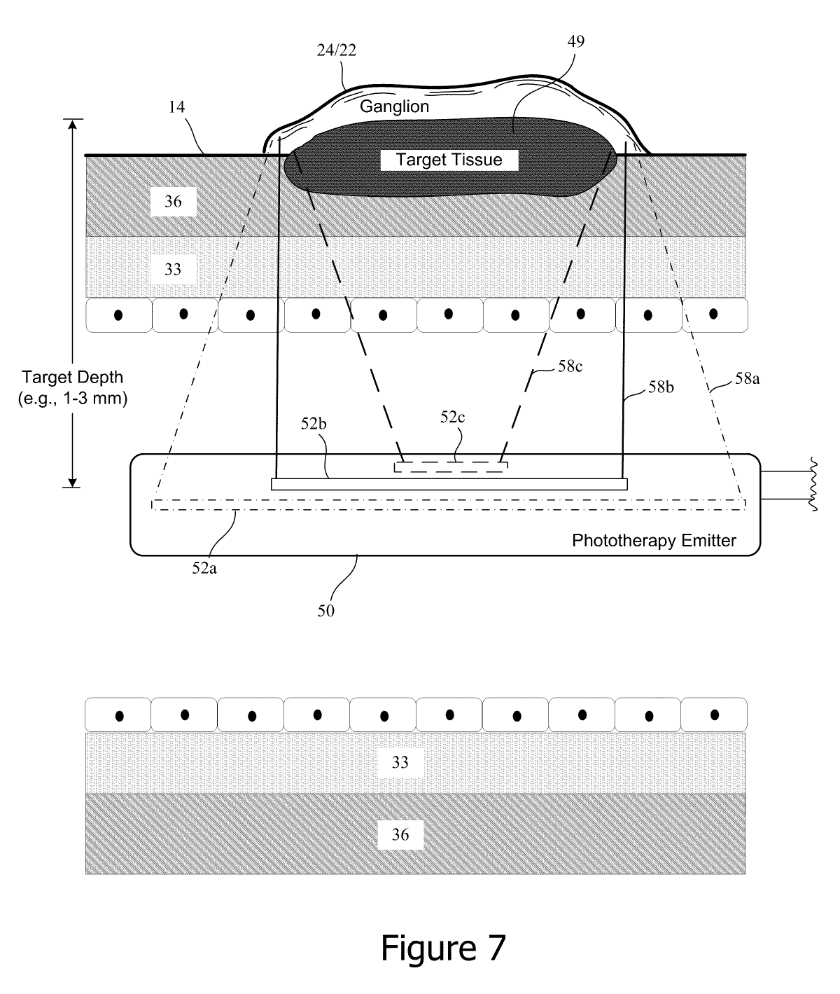

FIG. 7 illustrates an embodiment of a phototherapy treatment arrangement for denervating renal vasculature that contributes to renal sympathetic nerve activity in accordance with the invention. According to this embodiment, a phototherapy unit 50 is shown deployed in a patient's renal artery 12 and equipped with a phototherapy emitter 52 that is configured to deliver focused optical energy to target tissue 49 that includes one or both of renal nerves 14 and renal or aortic ganglia 24/22. Various emitters 52a-52c are shown for illustrative purposes that have different beam patterns 58. The phototherapy unit 50 may incorporate one or more of the same or disparate emitters 52a-52c.

FIG. 8 illustrates an embodiment of a phototherapy treatment arrangement for denervating renal vasculature that contributes to renal sympathetic nerve activity in accordance with the invention. According to this embodiment, a phototherapy unit 50 is configured for deployment within a balloon 64. The phototherapy unit 50 is shown disposed at a distal end of a catheter 51 and situated within the balloon 64 at a relatively central location. When expanded, the balloon 64 contacts the inner wall of the renal artery and stabilizes the phototherapy unit 50 at a desired location and orientation within the balloon (e.g., central location oriented axially along the balloon's central axis).

The balloon 64 may be configured to allow blood flow within the renal artery to provide cooling of the artery wall during an ablation procedure. A perfusion balloon or a fluid diversion arrangement may be used to provide support and centering for the phototherapy unit 50 and perfusion of blood for cooling of the artery wall during laser ablation. In other embodiments, the phototherapy unit 50 may be incorporated into a balloon 64 which can be expanded to the internal diameter of the renal artery, so that the balloon blocks the flow of blood. In general, it is desirable to prevent or limit the amount of blood in the optical path when the laser light passes from the balloon 64 and into the vessel wall. An occlusion balloon with flushing ability, for example, may be used in combination with the balloon 64 arrangement.

The balloon 64 can be filled with a fluid that allows optical energy emitted from the phototherapy emitter 52 to pass through the fluidic medium and through the balloon before striking the renal artery wall. The fluid in the balloon 64 may be circulated with open or closed irrigation to keep the inner wall of the renal artery from being heated above 50° C., while the internal tissue and nerve/ganglion of the renal artery is heated above at least 50° C., to disrupt the nerve function while avoiding stenosis of the renal artery wall due to the response to thermal injury. The fluid in the balloon 64 is preferably optically “transparent” to the wavelength of the optical emitted by the phototherapy unit 50.

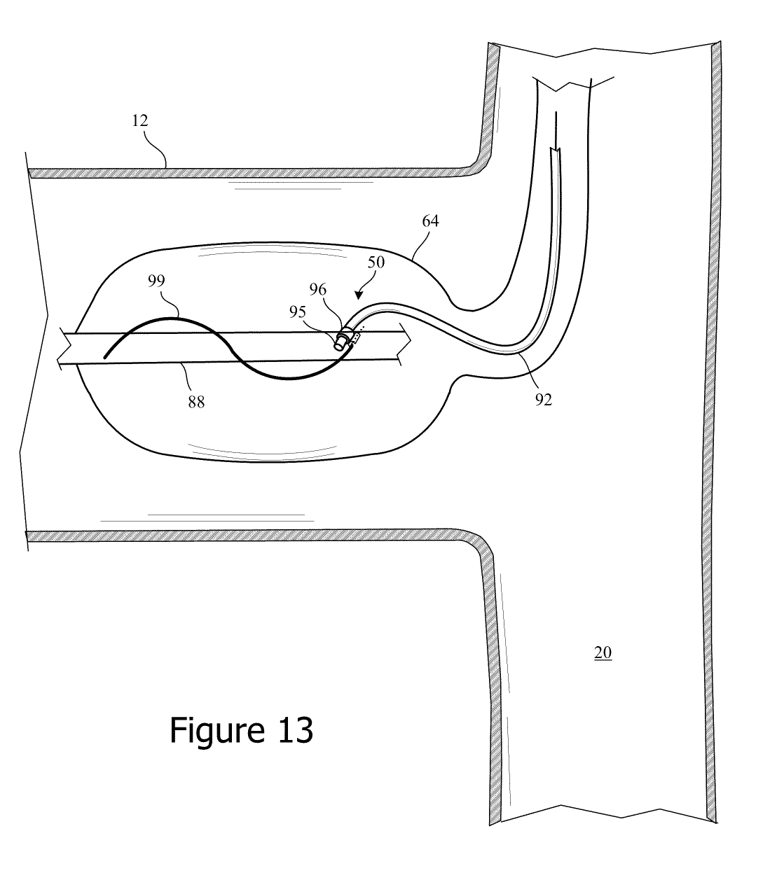

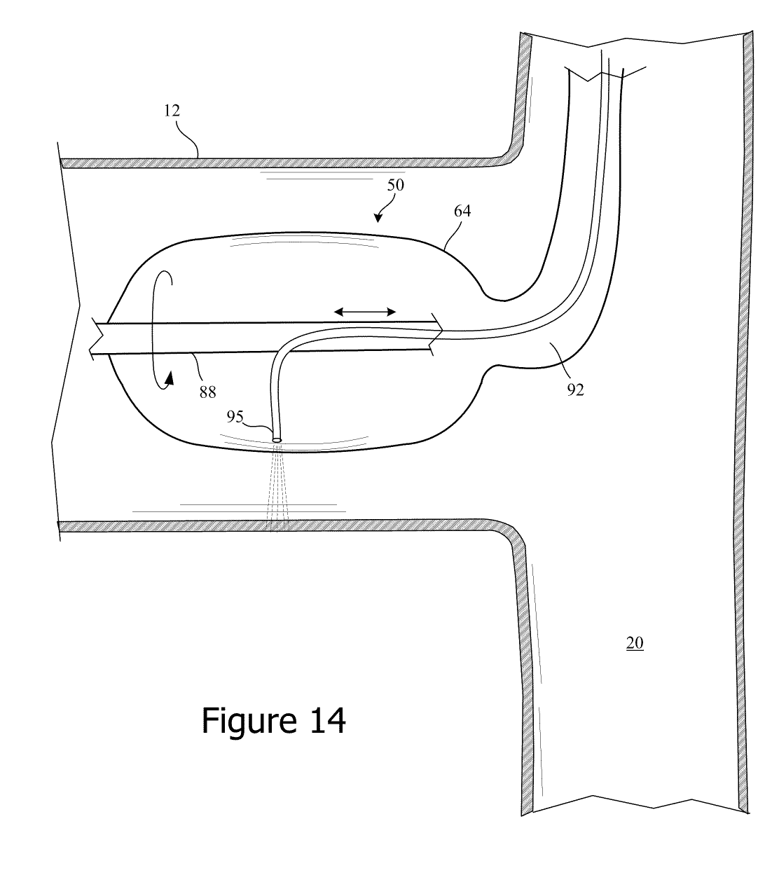

An advantage of using a balloon 64 of the type shown in FIG. 8 is that the phototherapy unit 50 can be translated and rotated without contacting the renal artery wall. In some embodiments, the shaft 67 can incorporate a spiral rail that forces the phototherapy unit 50 (or at least the emitter 52) to travel a helical path as it is advanced and retracted through the renal artery lumen. An illustrative example of such a configuration is shown in FIG. 13, which is described in detail hereinbelow. In other embodiments, the emitter 52 of the phototherapy unit 50 is oriented off-axis with respect to the longitudinal axis of the shaft 67. For example, the emitter 52 may be oriented at an angle of about 45° to about 135° relative to the longitudinal axis of the shaft 67, with about 90° representing a preferred orientation. An illustrative example of such a configuration is shown in FIG. 14, which is described in detail hereinbelow.

In various embodiments, the balloon 64 comprises a cryoballoon and the phototherapy unit 50 includes one or more phototherapy emitters 52. The cryoballoon 64 and phototherapy unit 50 cooperate to deliver optical and thermal energy to target tissue 49. In some embodiments, the phototherapy unit 50 comprises a phototherapy emitter 52 that creates lesions in the artery wall primarily through disruptive heating of target tissue. In other embodiments, the phototherapy unit 50 comprises a phototherapy emitter 52 that creates lesions in the artery wall primarily by production of multiple cavitation bubble at a multiplicity of foci.

The cryoballoon 60 shown in FIG. 8 includes an inlet manifold 61 and an outlet manifold 63 that facilitate pressurization, depressurization, and circulation of a cryogenic agent within the cryoballoon 64. The cryoballoon 64 comprises a single or multiple balloon structure, with appropriate lumens provided in the catheter 51 or other catheter of the treatment apparatus. One or more temperature sensors (not shown) are provided at the cryoballoon 60 to monitor temperature near or at the vessel wall.

In general, embodiments of the cryoballoon 64 may be implemented to deliver cryogenic therapy to cause renal denervation at therapeutic temperatures ranging between approximately 0° C. and approximately −180° C. For example, embodiments of a cryoballoon catheter may be implemented to deliver cryogenic therapy to cause renal denervation with temperatures at the renal nerves ranging from approximately 0° C. to approximately −30° C. at the higher end, and to about −140° C. to −180° C. at the lower end. Less robust renal nerve damage is likely for temperatures approaching and greater than 0° C., and more robust acute renal denervation is likely for temperatures approaching and less than −30° C., for example, down to −120 C to −180 C. These therapeutic temperature ranges may vary based on the combined therapeutic effect of delivering cryogenic and phototherapy energy to innervated target tissue of the renal artery and/or aorta.

According to another embodiment, a phototherapy unit 50 of the type shown in FIG. 8 (with or without a balloon, such as balloon 64) can include a lumen arrangement for transporting a thermal transfer fluid to provide local cooling (not freezing) of the intimal layer adjacent the phototherapy unit 50. In this embodiment, the catheter shaft 61 may incorporate one or more cooling lumens that interact directly with the adjacent intimal layer to counteract the application of higher intensity energies targeted for renal nerves that are further away or deeper in the artery wall.

The cyroballon 60 shown in FIG. 8 (and in other figures) is preferably a very low pressure balloon system. It is desirable to achieve minimal contact between the balloon 60 or other stabilizing arrangement and the inner wall of the renal artery in order to avoid injuring the sensitive endothelium of the artery. Very low pressure balloon systems can serve to provide minimal contact with the renal artery's inner wall and stabilization of the phototherapy unit 50.

The cryoballoon 60 or other stabilizing balloon can be constructed as a compliant balloon as is known in the art. For example, cryoballoon 60 may comprise a compliant material configured to enable the balloon 60 to inflate under a very low pressure, such as about 1 to 2 pounds per square inch (PSI) or less (e.g., 0.5 PSI or less) above an ambient pressure that is adjacent to and outside the balloon 60. The compliancy of cryoballoon 60 readily allows at least the ostial balloon 62 to conform to irregularities in the shape of the ostium 19 and surrounding tissue of the aortal/renal vasculature, which results in more efficient delivery of cryotherapy to the target tissue (i.e., renal nerve fibers and renal ganglia).

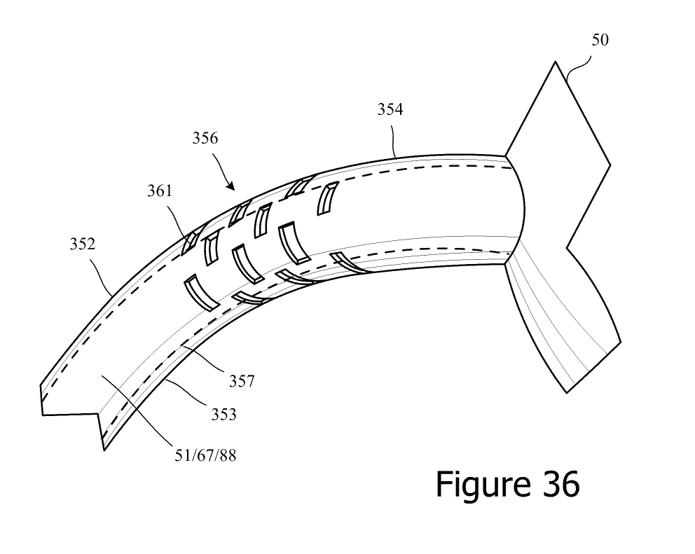

All or a portion of the cryoballoon 60 may be made of a highly compliant material that elastically expands upon pressurization. Because the cryoballoon 60 elastically expands from a deflated state to an inflated state, the cryoballoon 60 has an extremely low profile in the deflated state when compared to non-compliant or semi-compliant balloons. Use of high compliance materials in the construction of the cryoballoon 60, in combination with a hinge mechanism 56 built into the catheter 51 (see, e.g., hinge 356 shown in FIG. 36), provides for enhanced efficacy and safety when attempting to navigate a cryoballoon catheter 50 or other balloon of the present invention through a nearly 90 degree turn from the abdominal aorta 20 into the ostium 19 of the renal artery 12.

Suitable materials for constructing all or a portion of the cryoballoon 60 or other balloon include thermoplastic or thermoplastic elastomers, rubber type materials such as polyurethanes, natural rubber, or synthetic rubbers. The resulting balloon may be crosslinked or non-crosslinked. Other suitable materials for constructing all or a portion of the balloon 60 include silicone, urethane polymer, low durometer PEBAX, or an extruded thermoplastic polyisoprene rubber such as a low durometer hydrogenated polyisoprene rubber. These and other suitable materials may be used individually or in combination to construct the cryoballoon 60. Details of various materials suitable and configurations for constructing a cryoballoon 60 or stabilizing balloon are disclosed in commonly owned U.S. Pat. No. 7,198,632, U.S. patent application Ser. Nos. 12/980,952, and 12/980,972, which are incorporated herein by reference.

The cryoballoon 60 shown in FIG. 8 and other figures may be configured in accordance with those disclosed in commonly owned U.S. patent application Ser. Nos. 12/980,952, and 12/980,972, which are incorporated herein by reference. Embodiments of the invention may incorporate selected balloon, catheter, lumen, control, and other features of the devices disclosed in the following commonly owned U.S. patents and published patent applications: U.S. Patent Publication Nos. 2009/0299356, 2009/0299355, 2009/0287202, 2009/0281533, 2009/0209951, 2009/0209949, 2009/0171333, 2008/0312644, 2008/0208182, 2008/0058791 and 2005/0197668, and U.S. Pat. Nos. 5,868,735, 6,290,696, 6,648,878, 6,666,858, 6,709,431, 6,929,639, 6,989,009, 7,022,120, 7,101,368, 7,172,589, 7,189,227, 7,198,632, and 7,220,257, which are incorporated herein by reference. Embodiments of the invention may incorporate selected balloon, catheter, and other features of the devices disclosed in U.S. Pat. Nos. 6,355,029, 6,428,534, 6,432,102, 6,468,297, 6,514,245, 6,602,246, 6,648,879, 6,786,900, 6,786,901, 6,811,550, 6,908,462, 6,972,015, and 7,081,112, which are incorporated herein by reference.

According to various embodiments, a denervation therapy procedure using the apparatus shown in FIG. 8 involves selectively freezing and heating (and optionally thawing) target tissue that includes renal nerves and/or ganglia. For example, target innervated tissue is frozen using the cryoballoon 64. Before the target tissue thaws, ultrasonic energy is transmitted to the target tissue to fracture the renal nerve fibers and nerve sheaths located in the adventitia or vasa vasorum externae, thereby permanently terminating renal sympathetic nerve traffic along to treated renal nerves. A detailed discussion of renal nerve structures and degrees of nerve disruption that can be achieved in accordance with embodiments of the invention is provided in commonly owned U.S. Provisional Patent Application Nos. 12/980,952 and 12/980,972.

The cryoballoon 60 and/or catheter apparatus is preferably configured to allow blood to flow at or near the inner vessel wall after cryotherapy has been delivered to allow for local heating of the endothelium and adjacent tissue (e.g., intima and media tissue) while the adventitia remains frozen. Optical energy is preferably transmitted to the still-frozen adventitia layer (at least to deeper layers near the vessel's outer wall) to permanently disrupt renal nerves and ganglia included in the frozen tissue.

An advantage of using combined cryogenic and photo therapies for denervating renal artery and aortal tissue is that blood coagulation and embolization associated with RF ablation is avoided. Another advantage is that nerve regeneration over time that can occur when using cryotherapy alone is prevented, because of the fracturing of the renal nerve sheath resulting from mechanical disruption (e.g., microbubble implosion/explosion), when using cavitational phototherapy, or thermal coagulation (collagen reformation or fat rendered from the tissue), when using thermal phototherapy, which permanently disrupts renal nerve sheaths.

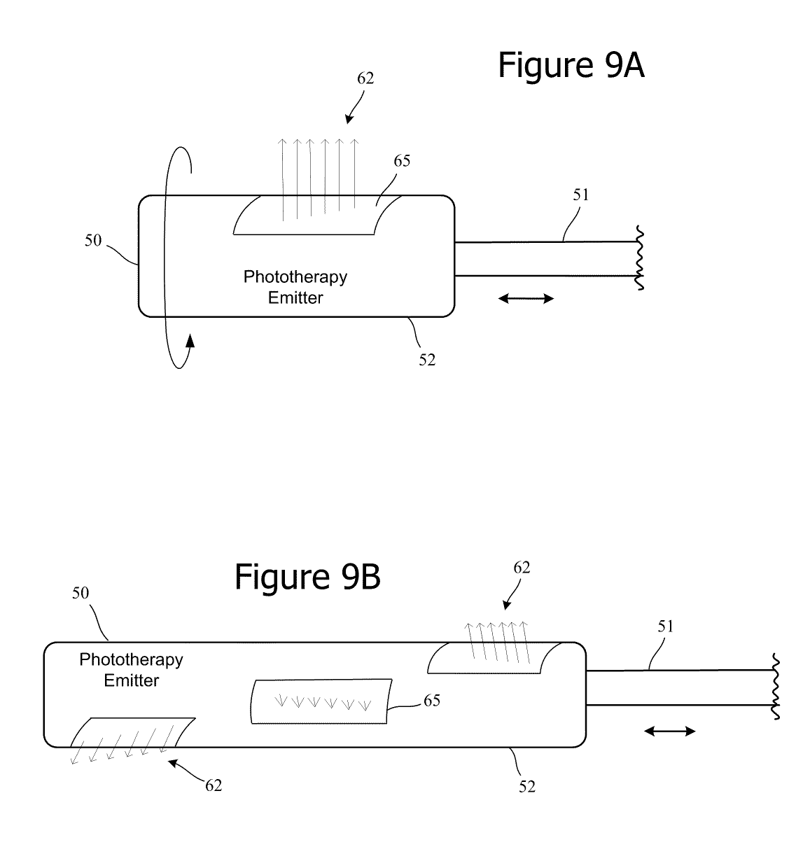

FIGS. 9A and 9B show different embodiments of a phototherapy treatment arrangement for denervating renal vasculature that contributes to renal sympathetic nerve activity in accordance with the invention. In the embodiment shown in FIG. 9A, the emitter 52 of the phototherapy unit 50 includes an aperture 65 through which an optical energy beam 62 passes. The aperture 65 may be a void or a material that allows for efficient transmission of the optical energy beam 62 from the emitter 52 and out of the phototherapy unit 50. The aperture 65 and emitter 52 are situated at a desired location of the phototherapy unit 50, and can be “aimed” at target tissue by rotating and translating the catheter 51 to which the phototherapy unit 50 is attached.

FIG. 9B shows a phototherapy unit 50 comprising a multiplicity of apertures 65 and emitters 52. The apertures 65 and emitters 52 are preferably situated so that their beam patterns 62 collectively impinge on renal artery tissue in a generally spiral pattern and at target depths in the renal artery wall. The spiral lesion may either be continuous or a sequential and overlapping line of ablated spots.

The phototherapy unit 50 shown in FIG. 9B advantageously facilitates a “one-shot” denervation therapy of the renal artery or other vessel in accordance with embodiments of the present invention. The term “one-shot” treatment refers to treating the entirety of a desired portion of a vessel without having to move the treatment implement or arrangement to other vessel locations in order to complete the treatment procedure (as is the case for a step-and-repeat denervation therapy approach). A one-shot treatment approach according to the embodiment shown in FIG. 9B advantageously facilitates delivery of denervation therapy that treats at least one location of each nerve fiber extending along a target vessel, such as the renal artery, without having to reposition the phototherapy unit 50 during denervation therapy delivery. The embodiment of a phototherapy unit 50 shown in FIG. 9B allows a physician to position the phototherapy unit 50 at a desired vessel location, and completely treat the vessel without having to move the phototherapy unit 50 to a new vessel location.

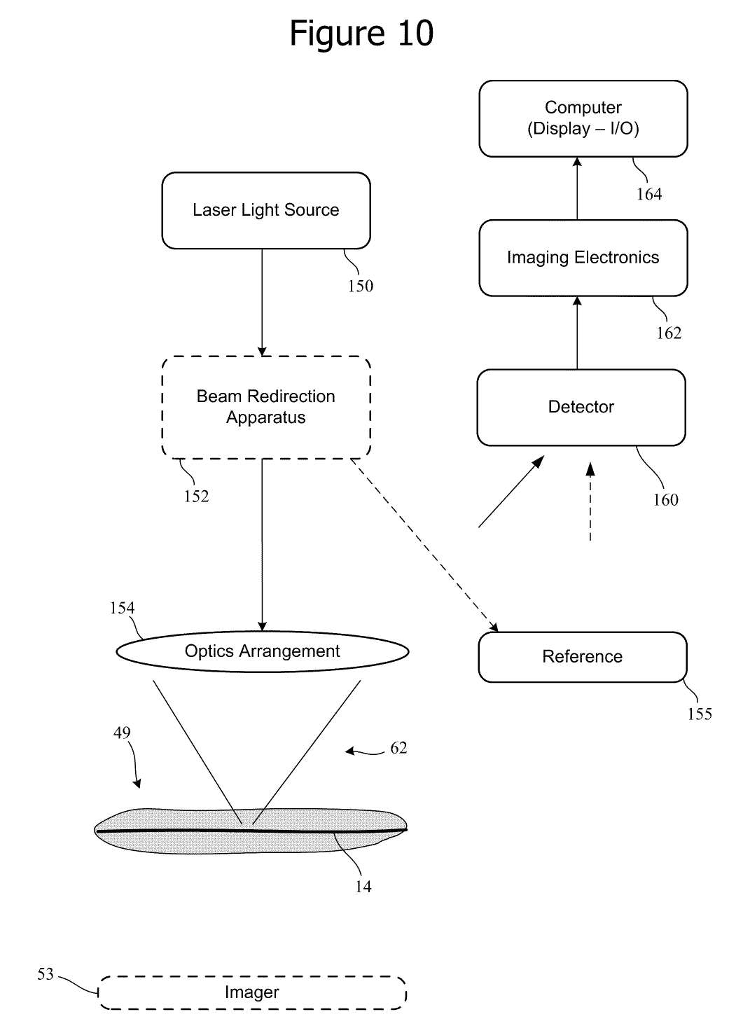

FIG. 10 illustrates an embodiment of a phototherapy treatment arrangement for denervating innervated vasculature that contributes to renal sympathetic nerve activity in accordance with the invention. FIG. 10 shows a phototherapy apparatus which includes a laser light source 150 that generates laser light having a desired wavelength and intensity. In some embodiments, the laser light source 150 is configured to generate a continuous wave (CW) light beam. In other embodiments, the laser light source 150 is configured to generate pulses of light. For example, the laser light source 150 may be configured as an ultrashort or ultrafast laser that produces tightly focused pulses of light.

According to various embodiments, phototherapy using laser light involves the conversion of laser light into heat when the incident laser beam is absorbed by target tissue. Irradiation of target tissue that includes nerves and ganglia with laser light, for example, leads to thermal damage of the target tissue. The diffusion of heat energy into the surrounding tissue, however, can thermally damage tissue outside the target area or volume of tissue. According to embodiments that utilize thermal laser/tissue interaction, local cooling apparatuses are preferably used to minimize thermal trauma to the surrounding tissue. Various cooling apparatuses are contemplated herein for this purpose, including cryoballoons, cryocatheters, cooling tips, irrigating tips, peltier cooling apparatuses, and blood diversion apparatuses, among others.

In accordance with other embodiments, potentially adverse complications that can result from heating tissue surrounding target tissue subjected to thermal laser phototherapy can be avoided. According to various non-thermal laser phototherapy embodiments of the invention, a short-pulsed laser is used to generate ultrashort light pulses in the picosecond to femtosecond range. Neural tissue, such as renal/aortal nerves and ganglia, can be ablated very precisely without causing any significant thermal damage to the surrounding tissue.

Ultrashort lasers and optics arrangements of the present invention can be configured to provide different types of photothermal and electromechanical interaction modes. In some embodiments, for example, ultrashort laser pulses are used to cause electromechanical disruption within target tissue. Because mechanical effects dominate in this interaction mode, and because ultrashort pulse durations do not allow time for the conduction of heat to the surrounding tissue, tissue structures surrounding the target tissue are not subject to injurious heating.

Suitable ultrashort lasers for use in the context of various embodiments of the invention include a Nd:YLF (neodymium:yttrium lithium fluoride)-laser system, an OPG/OPA (optical parametric generation/optical parametric amplification) laser system), and a Ti:sapphire (titanium-sapphire)-laser system, for example. Each of these laser systems may include an oscillator stage for pulse formation and a regenerative amplifier.

The phototherapy apparatus shown in FIG. 10 (and in other figures) can be operated in a scanning or imaging mode, a phototherapy delivery mode, or both. In some embodiments, it may be desirable to incorporate a separate phototherapy apparatus for each of a scanning or imaging mode and a phototherapy delivery mode, although each may receive light from a common laser light source 150.

The laser light source 150 may include one or more lasers and various polarizers that can be placed in the optical path or paths. According to various embodiments, light produced by the laser light source 150 is directed to an optics arrangement 154. The optics arrangement 154 includes one or more lenses, prisms, elements, and/or mirrors for shaping and directing light received from the laser light source 150 to target tissue 49, such as renal artery tissue which includes a renal nerve 14. The laser light exiting the optics arrangement 154 and penetrating the target tissue 49 is preferably a focused light beam 62 of sufficient energy to permanently disrupt renal nerves 14 included in the target tissue 49. An imager 53 (external or internal) is preferably used to facilitate positioning of the phototherapy treatment arrangement, and may also be used to determine or adjust various optical parameters, such as beam shape, direction, axial depth, longitudinal resolution, and beam intensity, for example.

By way of background, target renal artery tissue 49 can be heated using focused laser light 62, and, if the artery wall tissue temperature exceeds 50° C., the tissue can be killed. However, the target tissue 49 will not be physically and permanently disrupted until the temperature of the target tissue 49 exceeds about 65° C., where the collagen reforms. With focused light beams 62, a very small focus can be achieved deep in target tissues 49, such as a focal region or volume within the adventitia tunica or vasa vasorum that includes a renal nerve or ganglion. When the temperature within the target tissue 49 reaches a sufficient level (e.g., >65° C.), the target tissue 49 is thermally coagulated. By focusing at more than one tissue location or by scanning the focused beam, a volume of target tissue can be thermally ablated.

The optics arrangement 154 may include one or a number of optical and structural components. For example, a variety of lenses, prisms, and/or elements may be used in the context of various embodiments of the invention, including simple and compound lenses; objective, collimating, axicon, cylindrical, toroidal, and/or conical lenses or prisms; diffractive optical elements (DOE); and holographic optical elements (HOE), among others.

In accordance with other embodiments, light produced by the laser light source 150 may be used for imaging tissues of the renal and aortal vasculature. In laser imaging applications, the intensity of the laser light is preferably less than that required for ablation, and is preferably low enough to avoid thermal injury to scanned tissue. In the embodiment shown in FIG. 10, light produced by the laser light source 150 is directed to a beam redirecting apparatus 152, such as beam splitter. A probe beam 62 is directed from the beam redirection apparatus 152 to the optics arrangement 154, and penetrates and illuminates the target tissue 49. A reference beam is directed from the beam redirection apparatus 152 to an optical reference 155, such as a mirror apparatus.

Light backreflected from the target tissue 49 and light returning from the optical reference 155 is received at the detector 160. Imaging electronics 162, which may include optoelectronic components, preferably implements one or more known techniques for imaging scanned tissue 49 at various depths and transverse lengths or regions using the backreflected and return reference beams, including heterodyne, homodyne, and imaging interferometric techniques. Output from the imaging electronics 162 is received by a computer 164 which preferably includes a display. Data and visual information concerning the scanning and phototherapy procedures are preferably presented on the display. The computer 164 may include an interface (I/O) for communicating with other systems and devices.

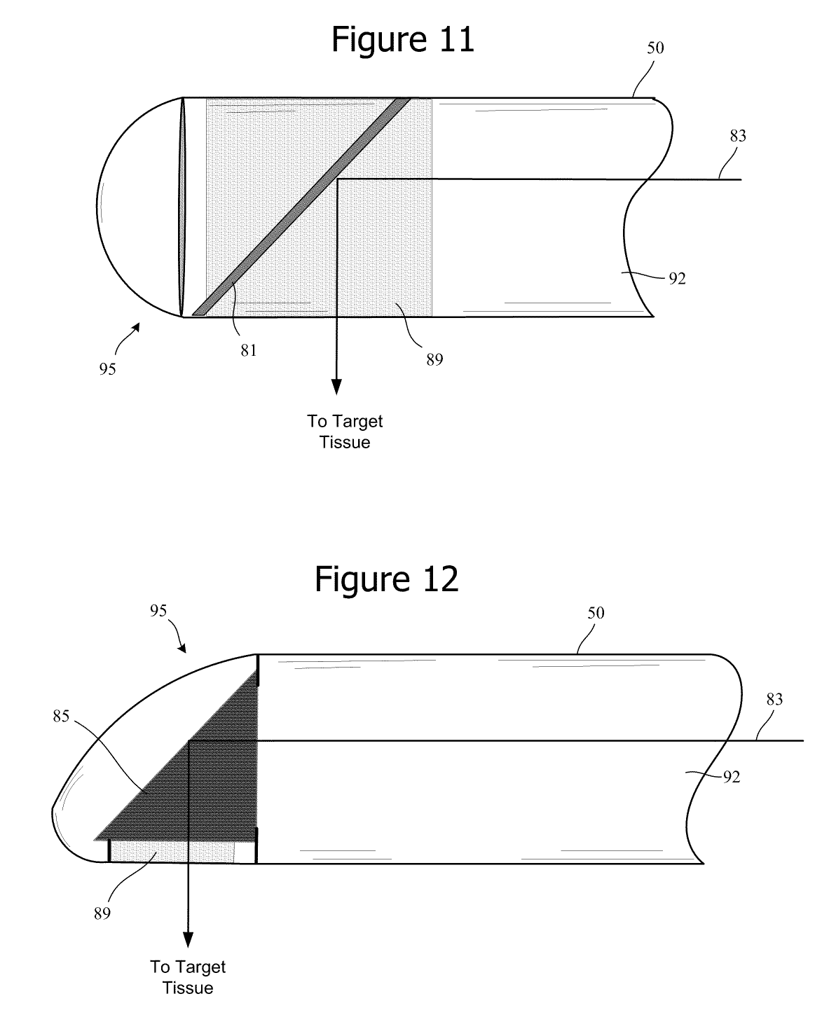

FIGS. 11 and 12 illustrate various phototherapy arrangements that can be deployed within innervated vasculature in accordance with embodiments of the invention. The phototherapy units 50 shown in FIGS. 11 and 12 use optical power from a laser to create an ablation at target nerves and ganglia of the renal artery, and of the abdominal aorta proximate the renal artery.

In the embodiment shown in FIG. 11, laser light power is conducted by an optical fiber 92 (which may be an optical fiber cable) to a tip assembly 95 at or adjacent a distal end of the optical fiber 92 of a phototherapy unit 50, which can be positioned in the renal artery. The tip assembly 95 includes a mirror 81 situated at or near the tip of the optical fiber 92. A front or back surfaced mirror 81 may be used, but a front surfaced mirror 81 is preferred because of the very high optical power density present.

The mirror 81 is preferably situated within the tip assembly 95 so that it deflects the laser light 83 conducted by the optical fiber 92 at a right angle relative to a longitudinal axis of the optical fiber 92. An optical lens 89 can be positioned within the tip assembly 95, which focuses the divergent light to the region of the renal artery wall which is to be ablated. The optical lens 89 may have a full or partial cylindrical configuration.

FIG. 12 shows another embodiment of a phototherapy arrangement in accordance with the invention. In the embodiment of FIG. 12, a TIR (total internal reflection) prism 85 is situated in the tip assembly 95 and is arranged to deflect the laser light 83 conducted by the optical fiber 92 at a right angle relative to a longitudinal axis of the optical fiber 92. An optical lens 89 can be positioned within the tip assembly 95 to focus the divergent light to the region of the renal artery wall which is to be ablated. If optical defects are kept sufficiently low, a TIR prism 85 may be a good alternative to the mirror 81 shown in FIG. 11.

The divergence angle of the laser beam 83 exiting the optical fiber 92 depends on the refractive index of the optical fiber core and the medium into which the beam 83 exits. Plastic optical fiber is generally not suitable for high power densities and also has a large numerical aperture which means that the optical power is easy to introduce into the fiber but will spread out at a wide angle fan when it exits the fiber. Glass and quartz optical fiber have narrow numerical apertures, meaning that the light is more difficult to couple into the entrance and the exit fan has a smaller beam divergence angle. It is noted that, if the beam divergence angle is small enough, no additional lens 89 may be required to collimate the beam 83. In many implementations of a phototherapy unit 50, however, a collimating lens 89 may be required. A suitable lens 89 can be in the form of a glass cylinder with a refractive index which varies with diameter so that it functions as a lens.

The laser source coupled to the phototherapy unit 50 may include a continuous wave laser. Visible and near-infrared (NIR) wavelengths are preferred, which are typical of argon ion, diode, Nd:YAG, and CO₂ lasers. Holmium and erbium lasers are also useful because of their NIR wavelengths.

The optical fiber 92 of a phototherapy unit 50 of the present invention may be of glass for wavelengths near the visible, but may require quartz for wavelengths in the near IR. It is noted that, at present, it may not be feasible to use CO₂ laser sources because there are currently no suitable materials for conducting the 10.6 micron infrared wavelength, despite the enormous power available at that wavelength. Developments in optical fiber technology may allow for future use of CO₂ laser sources.

The wavelength of the light of a phototherapy unit 50 in accordance with embodiments of the invention is preferably selected so that optical energy is absorbed substantially in the wall of the renal artery, preferably the outer wall region. In some embodiments, the phototherapy unit 50 is configured to emit laser light of sufficient power to raise the temperature of renal artery wall tissue to above 50° C. to kill the target artery tissue and nerve/ganglion within it. In other embodiments, the phototherapy unit 50 is configured to emit laser light of sufficient power to raise the temperature of the renal artery wall to above 65° C. to reform the collagen in target artery wall tissue and mechanically change the tissue property. In further embodiments, the phototherapy unit 50 is configured to emit laser light of sufficient power to raise the temperature of the renal artery wall tissue to between 65° C. and 100° C. to render the fat from the target tissue, and totally disrupt the target tissue and prevent chronic recovery of the nerve fibers/ganglion from the burn injury.

The tip assembly 95 of the phototherapy unit 50 shown in FIGS. 11 and 12 (and in other figures) is configured so that it can be rotated and translated longitudinally within the lumen of the renal artery to create a spiral ablation at target depth in the renal artery wall. The spiral lesion may either be continuous or a sequential and overlapping line of ablated spots. As previously discussed, it may be desirable to “cut” the renal nerve by thermal damage as the phototherapy unit 50 passes along the length of the renal artery, to avoid or minimize the risk of stenosis that may occur when ablating the renal artery in a circular fashion. Various embodiments of a phototherapy unit 50 in accordance with the invention contemplate ablating a spiral shape in the renal artery which circles the artery at least once as the burned area spirals and translates along the artery wall. As is also discussed herein, it may be desirable to ablate renal nerves 14 in a circular fashion, such as by ablating a circumferential shape in the renal artery which circles the artery at least once, without incurring undue risk of stenosis of the artery.

A support arrangement is preferably provided to center the tip assembly 95 within the renal artery so that the intensity of exiting light is approximately constant at the artery wall as the tip assembly 95 translates and rotates (see, e.g., stabilizing arrangement 55 shown in FIG. 6 and balloons 64 in FIGS. 8, 13, and 14). It is desirable to keep the optical power source of the tip assembly 95 relatively centered within the artery, so that the intensity of the laser beam does not vary significantly with location, which could result in over-treated and under-treated regions.

One approach to keeping the tip assembly 95 at a constant distance from the wall of the renal artery is to incorporate the tip assembly 95 into a balloon which is expanded until it fills the arterial lumen. Representative balloons are described above, such as very low pressure balloons.

In various embodiments, the maximum temperature of the inner wall of the renal artery may be kept below some target temperature, such as 50° C., by providing heat transfer sufficient to limit the temperature rise at the inner artery wall, while allowing for a temperature increase above the target temperature within the artery wall tissue sufficient to permanently disrupt the renal nerve fibers/ganglia.

According to one approach, the phototherapy apparatus is configured to allow blood flow within the renal artery to provide cooling of the artery wall during laser ablation. A perfusion balloon (e.g., fluted balloon) or a fluid diversion arrangement provided at the distal end of a laser ablation catheter (e.g., longitudinal inlet/outlet ports or channels), for example, may be used to provide support and centering for the tip assembly 95 and perfusion of blood for cooling of the artery wall during laser ablation.

Alternatively, the tip assembly 95 may be incorporated into a balloon which can be expanded to the internal diameter of the renal artery, so that the balloon blocks the flow of blood. The balloon can be filled with gas or a transparent liquid such as saline, and the laser beam 83 passes through the medium and through the balloon before striking the renal artery wall. The liquid in the balloon may be circulated with open or closed irrigation to keep the inner wall of the renal artery from being heated above 50° C., while the internal tissue and nerve/ganglion of the renal artery is heated above 50° C., to disrupt the nerve function while avoiding stenosis of the renal artery wall due to the response to burn injury.

With reference to FIG. 13, there is shown an embodiment of a phototherapy unit 50 that includes a tip assembly 95 incorporated into a balloon 64. An advantage of using a balloon 64 of the type shown in FIG. 13 is that the translation and rotation of the tip assembly 95 may be accomplished by drawing the tip assembly 95 along a spiral rail 99 mounted on a central shaft 88 of the balloon 64. A keyed channel arrangement 96, for example, may be disposed at the distal end of the optical fiber 92 that receives and captures the spiral rail 99. With the tip assembly 95 moving axially along a spiral path defined by the rail 99 inside the balloon 64, no scraping of the renal artery wall 12 will occur.

As discussed above, the balloon 64 may be filled with gas or a transparent liquid, and the liquid within the balloon 64 should be transparent to the wavelength used, and should minimally absorb the optical power. Many suitable gases and liquids are available for filling the balloon 64 that are transparent to the wavelength of the laser light used and minimally absorb the optical power of the laser light. Suitable gases include CO₂, O₂, N₂O, and possibly N₂, Ar, and Kr. Suitable liquids include saline and D5W, for example. It is highly desirable that the fluid not be toxic and should be highly soluble in blood to minimize possible embolic damage if the fluid should leak out of the balloon 64. The liquid in the balloon 64 may be circulated with open or closed irrigation to keep the inner wall of the artery from being heated above 50° C. during the laser ablation procedure.

FIG. 14 illustrates a phototherapy unit 50, which includes a tip assembly 95 situated at a distal end of an optical fiber 92, incorporated into a balloon 64 in accordance with various embodiments of the invention. In the embodiment shown in FIG. 14, the tip assembly 95 at the distal end of the optical fiber 92 is oriented off-axis with respect to the longitudinal axis of the shaft 88. In FIG. 14, the tip assembly 95 is shown oriented about 90° relative to the longitudinal axis of the shaft 88. It is understood that other tip assembly orientations may be desirable.

For example, the tip assembly 95 may be oriented at an angle of about 45° to about 135° relative to the longitudinal axis of the shaft 88. Also, the light emitting end of the tip assembly 95 may be biased more toward the shaft 88 than the outer surface of the balloon 64. The tip assembly 95 may be configured to extend from and retract into the shaft 88 under user control, which may be of particular benefit when expanding and contracting the balloon 64. The shaft 88 and the tip assembly 95 may be translatable and/or rotatable within the balloon 64.

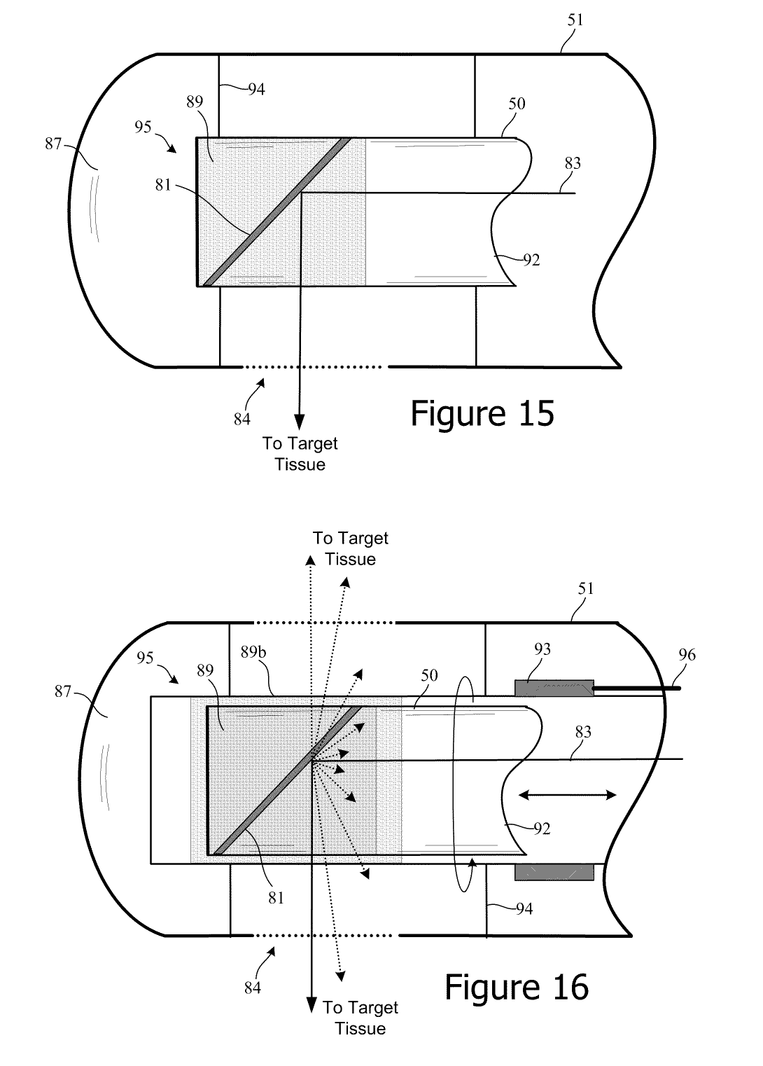

FIG. 15 shows a phototherapy arrangement which is incorporated at the distal end of a catheter 51 in accordance with embodiments of the invention. The distal end of the catheter 51 shown in FIG. 15 incorporates a tip assembly 95 of a phototherapy unit 50 which is supported by support members 94. The catheter 51 includes an aperture 84 through which the exiting light 83 passes. The aperture preferably comprises a material that is transparent to the wavelength used, and may incorporate or comprise the lens 89.

Support members 94 serve as a centering arrangement that maintains the tip assembly 95 at an axially centered orientation. The tip assembly 95 remains centered within the renal artery by properly positioning the distal end of the catheter 51 within the renal artery, typically by using a balloon such as that shown in other figures. In this manner, the intensity of light 83 exiting the tip assembly 95 is approximately constant at the artery wall as the tip assembly 95 translates and rotates within the lumen of the renal artery.

The catheter 51 may incorporate a channel arrangement (not shown), such as that shown in FIG. 13, disposed at its distal end. The channel arrangement may be configured to receive and capture a spiral rail 99, which allows the catheter 51 to move axially along a spiral path defined by the rail 99 inside the balloon 64.

FIG. 16 illustrates a phototherapy arrangement which is incorporated at the distal end of a catheter 51 in accordance with embodiments of the invention. According to this embodiment, the tip assembly 95 of a phototherapy unit 50 is rotatable within a lumen of the catheter 51, so that a circular lesion can be created at a desired depth in the wall of the renal artery. A circumferential section of the lumen wall of the catheter 51 proximate the circumferential aperture 84 may be a void or incorporate a material 89b that is transparent to the wavelength used (which may incorporate or comprise a lens).

The tip assembly 95 shown in FIG. 16 includes a movable mirror 81 that can be turned through approximately 360 degrees of rotation. One or more stops may be incorporated to limit rotation to 360 degrees or other predefined arc (e.g., <360°) to aid in preventing overtreatment of a circumferential region of the renal artery wall. A frictional element may be incorporated to enhance tactile feedback during tip assembly rotation.

In some embodiments, a drive wire 96 may be coupled to a drive mechanism 93 to facilitate manual rotation of the tip assembly, such as in a manner employed in an IVUS device. In other embodiments, the drive mechanism 93 may include a micromotor drive and receives electrical or pneumatic control signals from a control line 96, which is typically situated in a side lumen of the catheter 51. The tip assembly 95 may be configured for both rotation and translation in the embodiment shown in FIG. 16. A phototherapy apparatus according to FIG. 16 may be employed to deliver one-shot denervation therapy to nerves and ganglia of the renal artery and abdominal aorta.

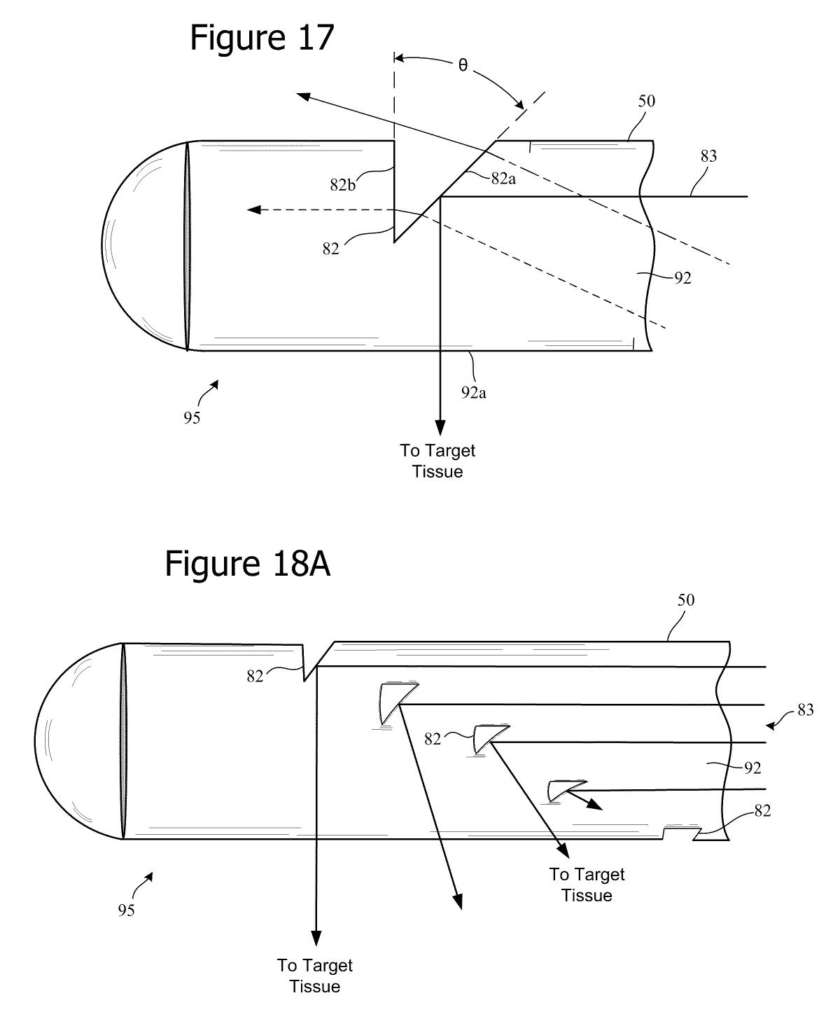

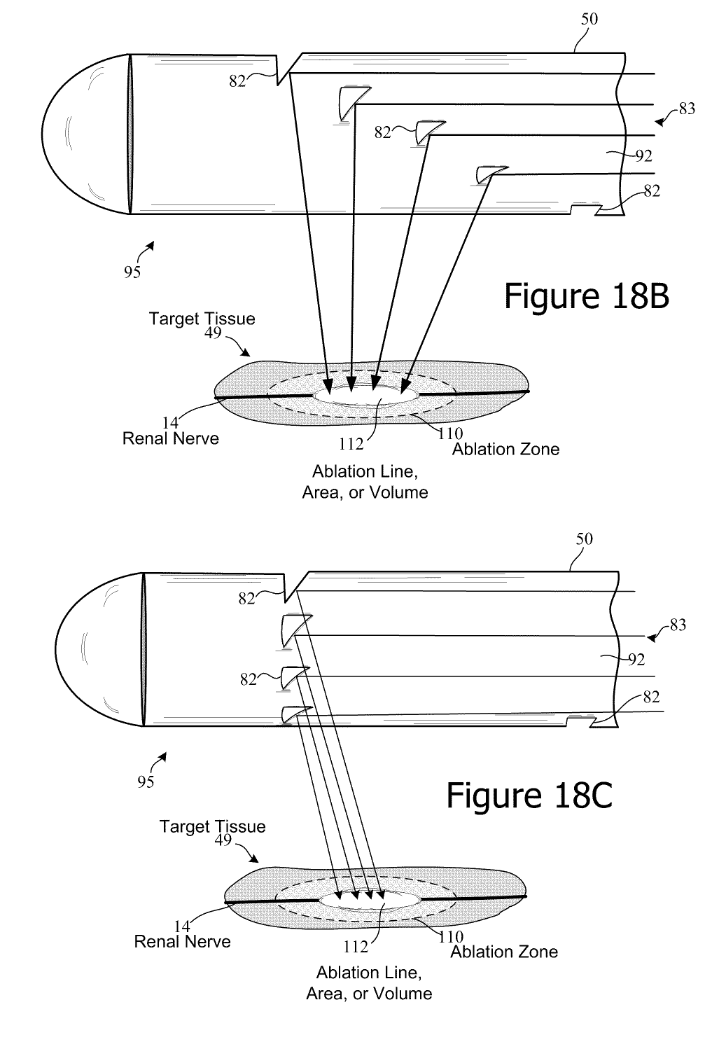

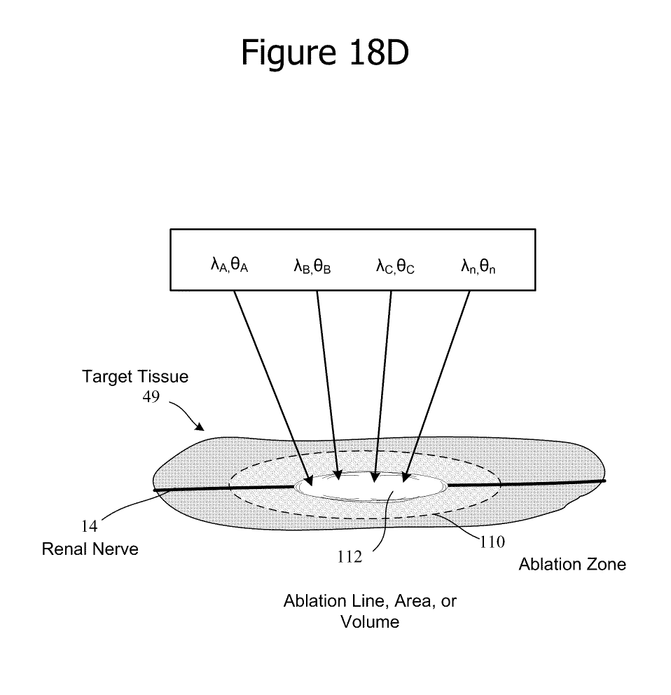

FIGS. 17 and 18A-18D illustrate tip assemblies 95 in accordance with various embodiments of the invention. FIG. 17 show a portion of an optical fiber 92 that incorporates one or more notches 82. Each notch 82 extends through the fiber cladding into the core material and defines a first surface 82a, inclined at an angle (e.g., 45°) to a plane normal to the longitudinal axis of the optical fiber 92, which acts as a reflection mirror such that a portion of the light propagated through the optical fiber 92 and impinging upon the surface 82a is reflected through the opposing wall 92a of the optical fiber 92. Each notch 82 also defines a second surface 82b, which is preferably normal to the axis of the optical fiber 92. The tip assembly 95 may be rotated within a lumen of the catheter 51 so that a circular lesion can be created at a desired depth in the wall of the renal artery.

By suitable design of the notch geometry, the proportion of light escaping via the notch 82 may be minimized, and the proportion reflected onto the opposing wall 92a maximized. If the reflectivity of surface 82a is enhanced (e.g., by silvering), then a greater proportion of the light striking surface 82a will be reflected onto wall 92a. The net effect of the notch 82 is therefore to divert a fixed proportion of the total light propagating through the optical fiber 92 out through wall 92a, with a much smaller proportion escaping through the notch 82 itself. The remaining light continues to propagate within the optical fiber 92, where it may impinge upon successive notches 82 (not shown), and at each notch, a further proportion of the light is diverted out through wall 92a.

FIG. 18A shows another embodiment of a tip assembly 95 that incorporates a multiplicity of notches 82 distributed in a circumferential and longitudinally spaced pattern that collectively complete at least one revolution of the distal end of the optical fiber 92. The multiplicity of notches 82 preferably form a spiral or helical pattern, providing for development of a continuous or a sequential and overlapping line of ablated spots that form a spiral shape in renal artery wall tissue.

FIG. 18B shows an embodiment of a tip assembly 95 that incorporates a multiplicity of notches 82 distributed in a circumferential and longitudinally spaced pattern. FIG. 18C shows an embodiment of a tip assembly 95 that incorporates a multiplicity of notches 82 distributed in a circumferential spaced pattern that collectively forms a circle or portion of a circle. The pattern of notches 82 shown in FIGS. 18B and 18C can, but need not, collectively complete a full circle about the distal end of the optical fiber 92. The multiplicity of notches 82 preferably direct light to a common focal site or zone, such as a line, area, or volume 112 within an ablation zone 110 that includes a renal nerve 14.

Directing a multiplicity of beams of the same or different wavelength from different angles (as shown in FIG. 18D) to a common site or zone aids in controlling scattering and increases the number of photons for absorption at the common site or zone. The tip assembly 95 of the phototherapy units 50 shown in FIGS. 18B and 18C can be rotated relative to the lumen of the renal artery so that a circular lesion is created at a desired depth in the wall of the renal artery.

In order to maintain a substantially uniform output light intensity along the notched region of the optical fiber 92, the spacing between successive notches 82 is decreased in the direction of intended light travel. Although the light emitted from each successive notch 82 decreases as a result of the leakage of light from preceding notches, this is compensated by increasing the notch density in the direction of light travel. Alternatively, the angle of inclination of the notch surface(s) may be changed or the cross-sectional area of each surface of successive notches 82 may be increased in the direction of intended light travel, so as to compensate for the aforesaid light loss. Additional details of a notched optical fiber 92 that can be adapted for use in a tip assembly 95 of the present invention are disclosed in U.S. Pat. No. 5,432,876, which is incorporated herein by reference.

The phototherapy units 50 shown in FIGS. 18A-18D advantageously facilitate a one-shot denervation therapy of the renal artery or other vessel in accordance with embodiments of the present invention. The phototherapy units 50 shown in FIGS. 18A-18D allow a physician to position the tip assembly 95 at a desired vessel location, and completely treat the vessel without having to longitudinally translate the tip assembly 95 to a new vessel location.

It is understood that the mirrors, prisms, and notches shown in FIGS. 15 and 18A-18D can be arranged to deflect laser light at angles other than right angles, including various acute and obtuse angles. For example, a phototherapy apparatus that includes a multiplicity of mirrors, prisms, notch features, or other mechanisms (or combinations thereof) can be arranged to deflect laser light at disparate angles relative to the longitudinal axis of the optical fiber.

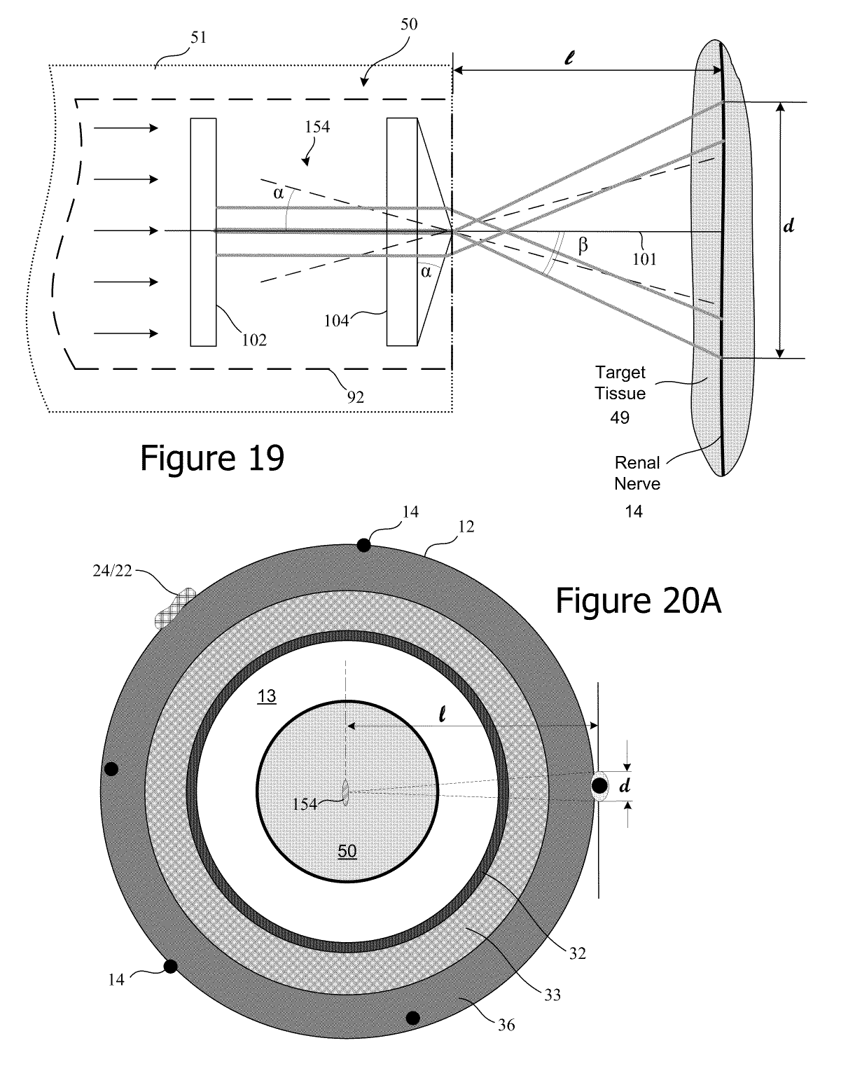

FIG. 19 illustrates an optics arrangement of a phototherapy unit 50 provided at a distal end of a catheter 51 for focusing laser light at target tissue in accordance with embodiments of the invention. The phototherapy unit 50 shown in FIG. 19 can be used to provide high transverse resolution ranging with a high depth of field for scanning target tissue 49 and for delivering ablative phototherapy to the target tissue 49. The phototherapy unit 50 incorporates an optics arrangement 154 that provides for the projection of focused optical energy into target tissue 49 at a desired depth and with a longitudinal aspect.

The optics arrangement 154 includes a collimating lens 102 and an axicon lens 104 disposed at or adjacent a distal end of an optical fiber 92. The optical fiber 92 is preferably supported within or on an elongated member, such as a catheter 51. In some embodiments, the optics arrangement 154 is physically connected to the optical fiber 92. In other embodiments, the optics arrangement 154 is physically separate from the optical fiber 92 and situated adjacent the portion of the optical fiber 92 through which laser light is emitted.

According to the embodiment shown in FIG. 19, the axicon lens 104 may be implemented as a conical lens or a rotationally symmetric prism. For example, the axicon lens 104 may comprise a reflective, transmissive, or diffractive optical element. An axicon lens arrangement can be used to construct a phototherapy unit 50 of the present invention that uses an axial line focus to achieve good resolution imaging over relatively large depths of field.

The axicon lens 104 shown in FIG. 19 converts a parallel laser beam produced from the collimating lens 102 into a ring shaped image that is projected to a desired focus depth within target tissue 49. The optics arrangement 154 of FIG. 19 advantageously projects laser energy oriented along an optical axis 101 into a volume of target tissue without adversely affecting (e.g., injuring) intervening tissue. The optics arrangement 154 illustrated in FIG. 19 can be used in one or both of scanning for target tissue 49 (e.g., innervated tissue) and ablating target tissue 49.

Those skilled in the art will understand that the axicon lens 104 can be designed to have a specified depth of focus, l, and transverse diameter (or length), d, which determines the spot or sphere size of the projected ring image. In FIG. 19, α is the angle formed by the conical surface within the flat surface of the axicon lens 104, and β is the intersection angle of the geometrical rays with respect to the optical axis 101. For a collimated Gaussian beam, for example, the spot size, d, may be determined by the following equation:

$$d = \frac{4 λ f}{π D}$$

where, D is the beam diameter at the axicon lens 104, f is the focal length of the lens 104, and λ is the wavelength. Additional details for implementing various optics arrangements 154 that include an axicon lens and other optics arrangements that can project optical energy into target tissue 49 at a desired depth in accordance with embodiments of the invention and in view of the “controlled scattering” considerations discussed previously of are disclosed in U.S. Pat. No. 7,310,150, which is incorporated herein by reference.

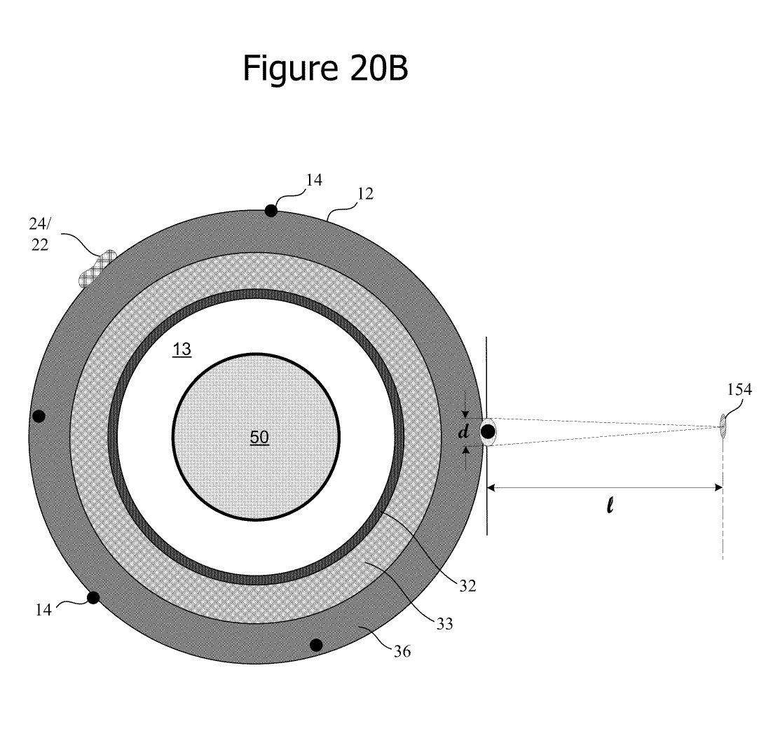

FIG. 20A is an exaggerated sectional view of a renal artery 12 and a laser beam emitted from an optics arrangement 154 of a phototherapy unit 50 positioned within a lumen 13 of the renal artery 12. FIG. 20B is an exaggerated sectional view of a renal artery 12 and a laser beam emitted from an optics arrangement 154 of a phototherapy unit 50 positioned at an extravascular or transvascular location proximate the outer wall of the renal artery 12. Access to the outer wall of the renal artery 12 may be achieved using an intra-to-extra vascular route via the renal vein or inferior vena cava, for example.

In the illustrative embodiments shown in FIGS. 20A and 20B, the optics arrangement 154 is configured to focus optical energy at a volume of tissue of the adventitia 36 proximate the outer wall of the renal artery 12 that includes a renal nerve 14. As previously discussed, the phototherapy unit 50 may be operated to scan the adventitia 36 and vasa vasorum for renal nerves 14 and ganglia 24/22 using relatively low power laser light. The phototherapy unit 50 may also be operated to deliver ablation phototherapy to create a lesion that permanently disrupts the target renal nerve 14. Employment of an optics arrangement 154 that includes an axicon lens 104 advantageously spares tissues of the intima 32 and media 33 from injury when using an intravascular phototherapy embodiment such as that depicted in FIG. 20A, and spares non-targeted tissues of the vasa vasorum and adventitia 36 from injury when using an extravascular or transvascular phototherapy unit embodiment such as that depicted in FIG. 20B.

It is generally known that a typical renal artery of a human adult has a diameter of about 5 to 6 mm. Embodiments of the optics arrangements 154 described herein may be implemented to project optical energy to a focal site located about 1 mm to about 5 mm from the optics arrangement 154, and preferably to tissue depths of about 1 mm to about 3 mm, and preferably to tissue depths of about 0.5 mm to about 2 mm, which is sufficient to reach target tissues of the renal artery wall, including ganglia, and the vasa vasorum proximate the outer wall of the renal artery from an intravascular, extravascular, or transvascular location.

In various embodiments, an optics arrangement 154 may be implemented to project optical energy to focal sites having a tissue depth that falls within a target range that encompasses the outer adventitial layers of the renal artery. In other embodiments, an optics arrangement 154 may be implemented to project optical energy to focal sites having a tissue depth that falls within a target range that encompasses the outer adventitial layers of the renal artery and the vasa vasorum proximate the outer wall of the renal artery. It is understood that optics arrangements 154 may be configured for projecting optical energy to one or more foci having tissue depths that fall within a target range that encompasses renal nerves of the renal artery's ostium and ganglia of the abdominal aorta.

According to various embodiments, a multiplicity of disparate optics arrangements 154 may be used to project optical energy to multiple foci having different target tissue depths. Particular ones of the multiplicity of disparate optics arrangements 154 may be selectively operated for scanning and/or ablating at different tissue depths. For example, scanning at different depths within the outer renal artery wall and adjacent vasa vasorum can be performed to locate target tissue that includes one or both of renal nerves and ganglia. After determining the target tissue location and depth, which may alternatively be accomplished using a separate internal or external imager, a selected one or more of the optics arrangements 154 may be used to ablate the target tissue at the determined depth.

A number of different optics arrangements 154 that provide for different target tissue depths and transverse diameters or lengths may be incorporated in or adjacent a multiplicity of optical fibers 92, or may be provided in a manifold, such as those shown in FIGS. 22A-22B, 23, and 24, which will be discussed hereinbelow. Other optics arrangements are contemplated, including those with optical properties that can be dynamically adjusted for purposes of imaging and ablating target tissue in the context of various embodiments of the invention. Such dynamically adjustable optical properties include target tissue depth, diameter or length of transverse resolution, wavelength of laser light received and/or emitted, beam direction and pattern, and optical energy transmission efficiency, for example.

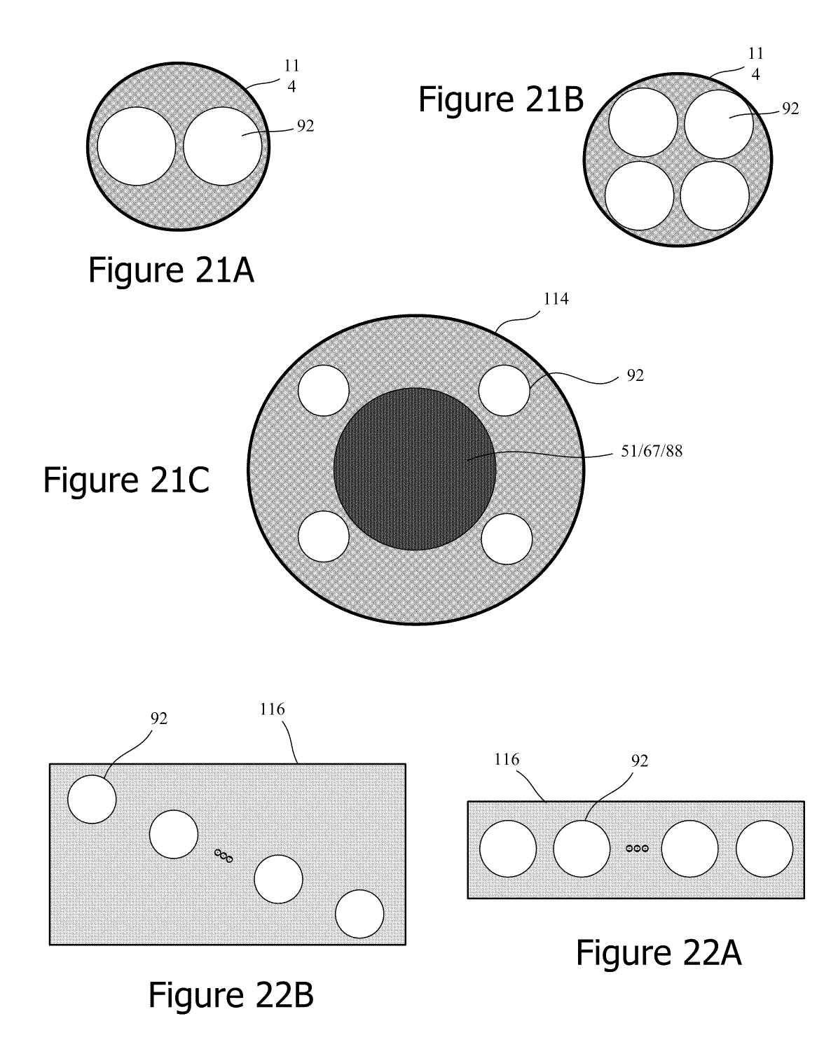

FIGS. 21A-22B illustrate various embodiments of a bundle 114 of optical fibers 92 that can be used to supply laser light to a phototherapy unit 50. FIG. 21A shows two optical fibers 92 that are included in the fiber bundle 114, while FIG. 21B shows four optical fibers 92. FIG. 21C shows four optical fibers 92 spaced around the shaft 51 of a catheter 51 or a shaft 67/88 of balloons 64 shown in FIGS. 8, 13, and 14, respectively. The four optical fibers 92 may be positioned within lumens of the catheter 51 or shaft 67/88, or on the exterior of the catheter 51 or shaft 67/88 (e.g., in sidewalls of the catheter 51 or shaft 67/88). A multiplicity of optical fibers 92 may be arranged in various configurations, including horizontal, vertical, or diagonal arrangements, for example, as is shown in FIGS. 22A and 22B. A manifold 116 that serves to support the optical fibers 92 in a desired configuration and/or orientation may be used.

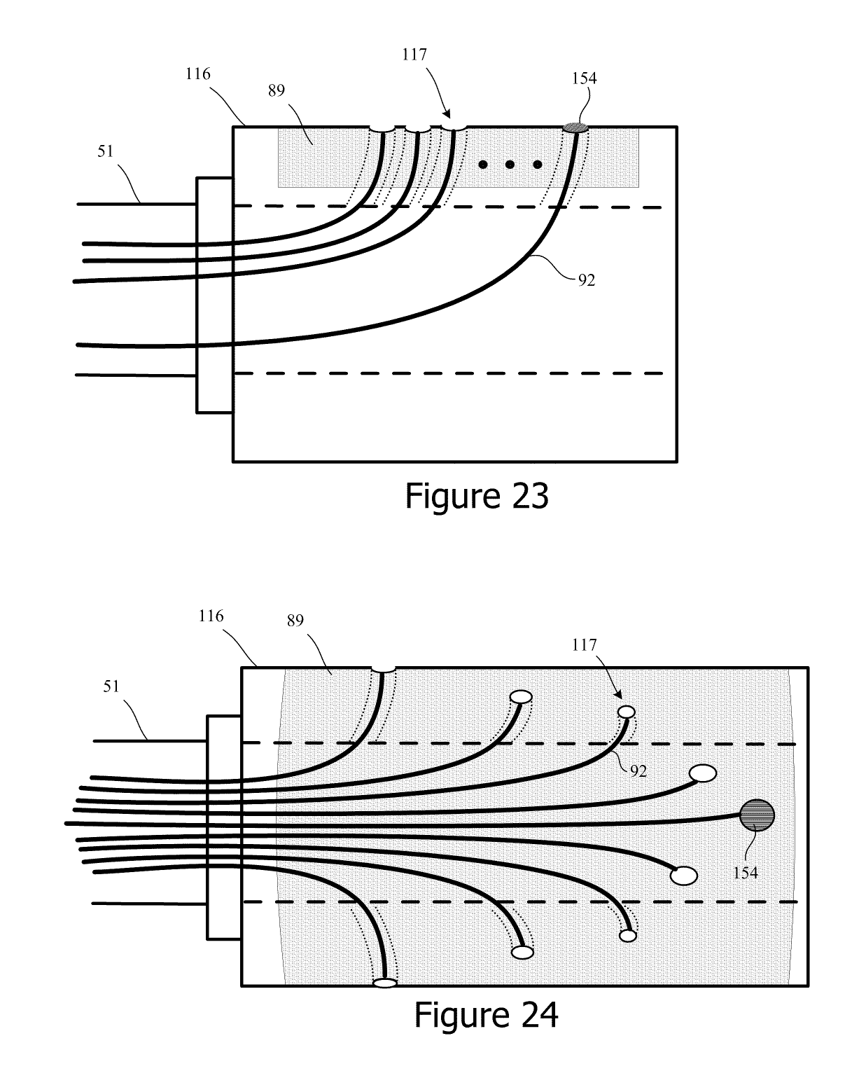

FIGS. 23 and 24 illustrate optical fiber manifolds 116 according to embodiments of the invention. In FIG. 23, a multiplicity of optical fibers 92 pass through one or more lumens of a catheter 51 to which a manifold 116 is coupled. Each of the optical fibers 92 is positioned within a bore 117 of the manifold 116. The orientation of each bore 117 is preferably selected to achieve a desired orientation of the distal end of each optical fiber 92. In some embodiments, an optics arrangement 154 of a type described herein is situated within each bore 117. In other embodiments, an optics arrangement 154 is situated at the distal tip of each optical fiber 92, which may be within, partially within, or adjacent the distal tip of the optical fiber 92. In further embodiments, one or more lenses 89 may be disposed at or proximate an outer surface of the manifold 116, in addition to or exclusive of other optics arrangements 154.

In the embodiment shown in FIG. 23, the distal ends of the optical fibers 92 are oriented substantially normal to a longitudinal axis of the manifold 116 and arranged in an axially spaced-apart relationship. The distal ends of the optical fibers 92 may be arranged in a relatively straight line or be circumferentially offset relative to the longitudinal axis of the manifold 116.

According to the embodiment shown in FIG. 24, the distal ends of the optical fibers 92 are oriented substantially normal to a longitudinal axis of the manifold 116 and arranged in an axially and circumferentially spaced-apart relationship. The distal ends of the optical fibers 92 are preferably arranged around the manifold 116 to facilitate scanning and ablation in a spiral pattern. Alternatively, the distal ends of the optical fibers 92 may be arranged around the manifold 116 to form a circle to facilitate scanning and ablation in a circular pattern.

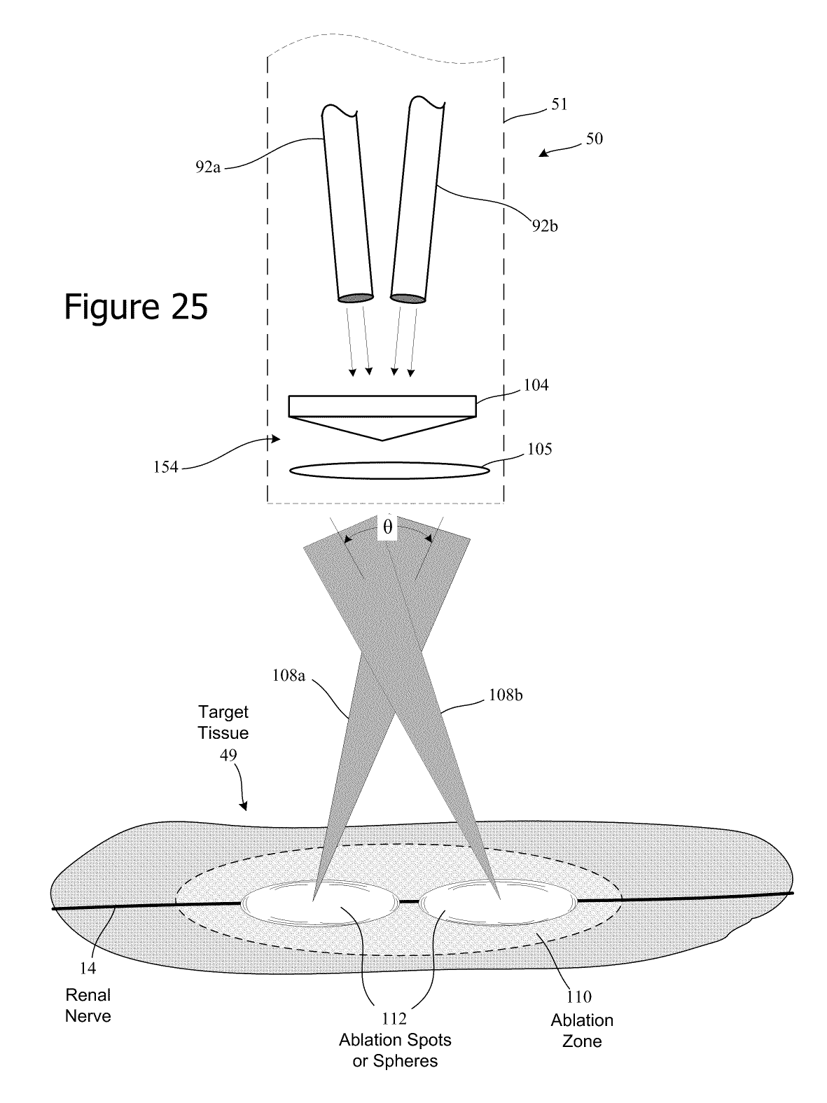

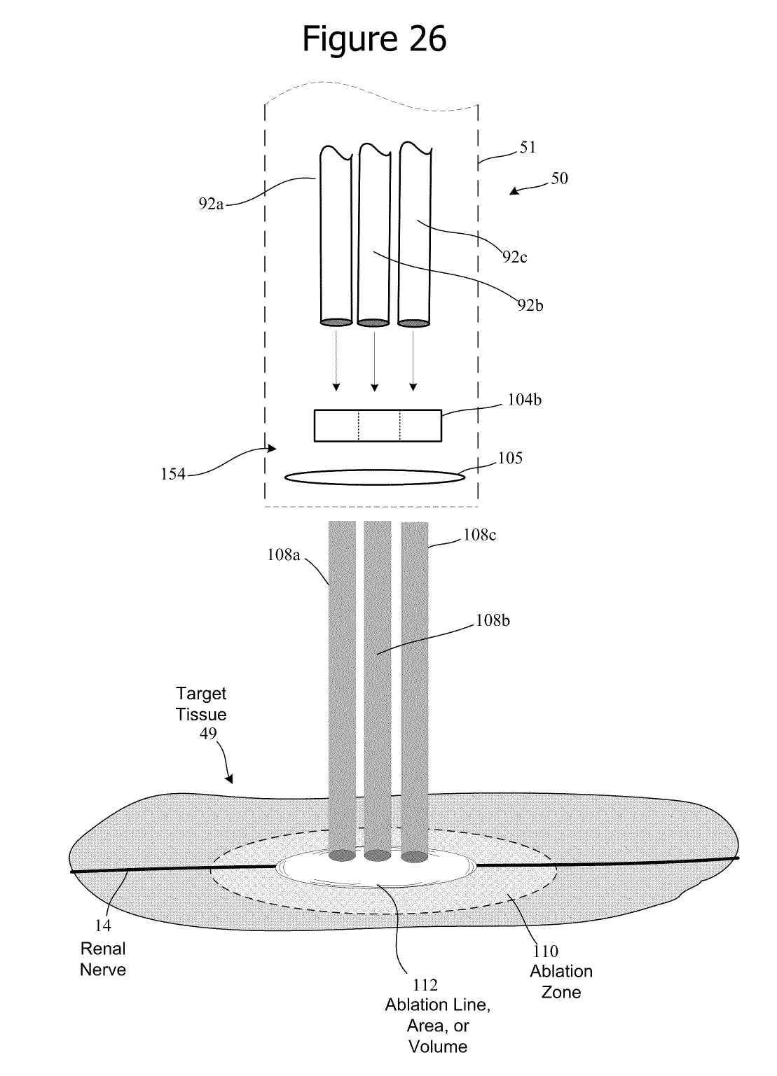

FIG. 25 illustrates a phototherapy unit 50 in accordance with embodiments of the invention. The phototherapy unit 50 shown in FIG. 25 includes a catheter 51 through which two optical fibers 92a and 92b extend and terminate at or proximate a distal tip of the catheter 51. The phototherapy unit 50 is shown to include an optics arrangement 154 that incorporates an axicon lens 104 and, if needed, one or more additional lenses, such as objective lens 105. It is noted that the ordering of lenses may differ from that shown in FIG. 25. The phototherapy unit 50 illustrated in FIG. 25 may be implemented for intravascular, extravascular, or transvascular deployment.

In this embodiment, the two optical fibers 92a and 92b are oriented in a non-parallel relationship, such that an angle is defined between the two optical fibers 92a and 92b relative to a longitudinal axis of the catheter's distal end. Laser light emitted from the two optical fibers 92a and 92b passes through the axicon lens 104 and objective lens 105 (if needed) to create two conical beams 108a and 108b having axially extended foci. Optical energy is projected to two target sites 112 within target tissue 49 to define an ablation zone 110.

In FIG. 25, the ablation zone is shown to include a renal nerve 14. Each of the target sites 112 represents an ablation spot or sphere having a transverse length or radius. The longitudinal spacing between the target sites 112 is a function of an angle, θ, formed between the two conical beams 108a and 108b. This longitudinal spacing is preferably selected to allow the two target sites 112 to effectively merge, so as to form a relatively large lesion in the target tissue 49 at a desired depth. It is noted that multiple optical fibers 92a-92n using the same or multiple optics arrangements 154 may be used that are co-parallel, rather than being oriented as shown in FIG. 25, but that the arrangement illustrated in FIG. 25 may provide for greater consistency in terms of ablation spot/sphere shape and size.

According to various embodiments, an ultrafast laser is used to generate a sequence of laser pulses of sufficient power to create ablations at the target sites 112. Suitable ultrafast lasers include femtosecond and picosecond lasers, for example. Femtosecond laser pulses are capable of targeting and cutting cellular and subcellular structures, such as cellular structures of nerves and ganglia, without damaging surrounding cells or tissue structures.

Laser pulses are preferably directed to target tissue 40 in a sequential manner. Each pulse creates plasma in a focal zone (ablation zone 110) within the target tissue 40 as a result of multiphoton absorption and impact ionization. At a certain density, this plasma becomes highly absorptive for the laser radiation, which results in rapid heating and subsequent explosive vaporization of the target tissue 49 within the ablation zone 110. At energies exceeding a vaporization threshold, this rapid heating and subsequent explosive vaporization produces a cavitation bubble formed around the focal volume at the target site 112 that mechanically ruptures the target tissue 49 within the ablation zone 110. The size of the resulting rupture zone sets the limit to maximum separation between subsequent pulses for producing a continuous lesion in the target tissue 49. A sequential laser pulse approach according to the embodiment shown in FIG. 25 provides for the creation of lesions in target tissue 40 having a three-dimensional shape at a desired depth, while leaving intervening tissue relatively undamaged.

Although a single optical fiber may be used to create lesions in target tissue 40 in a manner described above, embodiments having two optical fibers 92a and 92b as shown in FIG. 25 can be used to create a continuous cut in the target tissue 49 formed when rupture zones produced by separate cavitation bubbles at two target sites 112 coalesce. An extended zone of target tissue can be cut by simultaneous application of laser energy in multiple foci (two, three, four or more foci) using the apparatus shown in FIG. 25. Simultaneous formation of multiple cavitation bubbles results in hydrodynamic interactions that can lead to significant extension of the rupture zone in target tissue 49. In particular, two or more simultaneously expanding bubbles compress and strain tissue between the bubbles, while simultaneously collapsing bubbles can produce jets directed towards each other. Based on a measured tissue strain threshold, a deformation map of target tissue 49 can be generated, from which the rupture zone may be determined as a function of maximum bubble size and distance between the bubbles.

FIG. 26 illustrates a phototherapy unit 50 in accordance with other embodiments of the invention. The phototherapy unit 50 shown in FIG. 25 includes a catheter 51 through which three optical fibers 92a-92c extend and terminate at or proximate a distal tip of the catheter 51. The phototherapy unit 50 is shown to include an optics arrangement 154 that incorporates a cylindrical lens 104b and, if needed, one or more additional lenses, such as objective lens 105. The ordering of lenses may differ from that shown in FIG. 26. The phototherapy unit 50 illustrated in FIG. 26 may be implemented for intravascular, extravascular, or transvascular deployment.

In this embodiment, the three optical fibers 92a, 92b, and 92c are oriented in a parallel relationship, although a non-parallel arrangement may be used as discussed above. Laser light emitted from the three optical fibers 92a-92c passes through the cylindrical lens 104b and objective lens 105 (if needed) to create three cylindrical beams 108a, 108b, and 108c having axially extended foci. Optical energy is projected to a target site 112 within target tissue 49 to collectively define a somewhat diffuse ablation zone 110. The optical energy projected to the target site may produce one, two, or three bubbles that define an ablation spot or sphere having a transverse length or radius. In cases where two or three bubbles are produced, the longitudinal spacing between bubbles at the target site 112 is a function of longitudinal spacing between cylindrical beams 108a, 108b, and 108c. This longitudinal spacing is preferably selected to allow multiple bubbles at the target site 112 to effectively merge, so as to form a relatively large lesion in the target tissue 49 at a desired depth upon bursting.

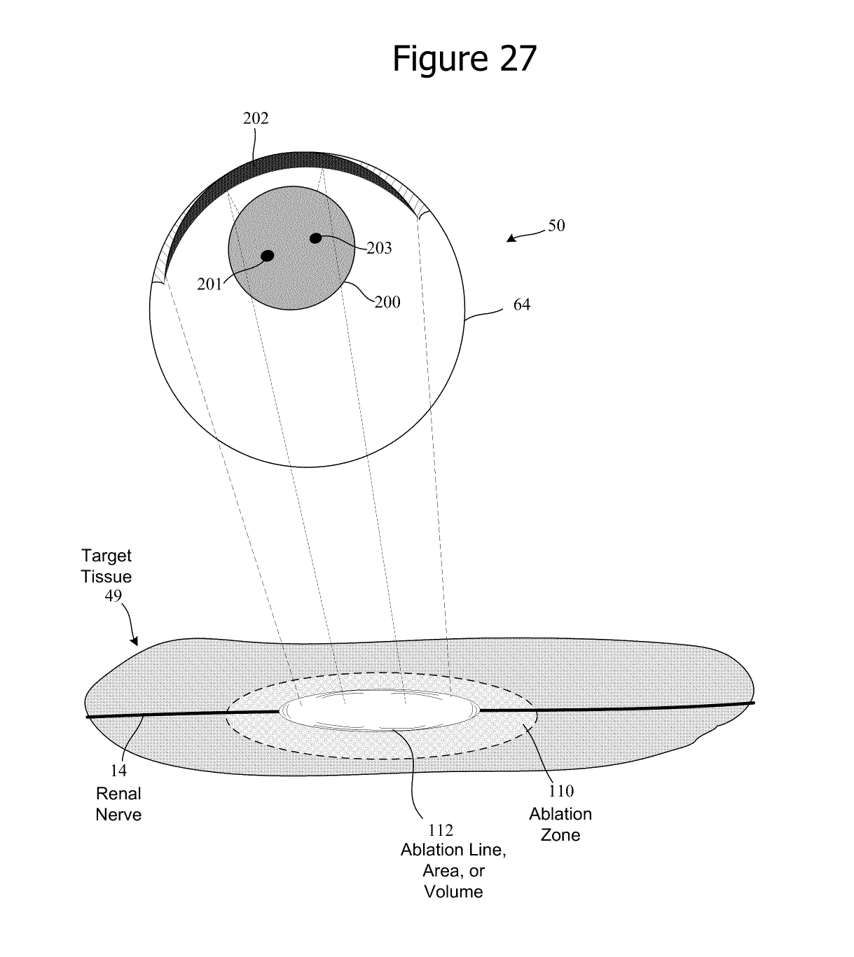

FIG. 27 illustrates a phototherapy unit 50 in accordance with other embodiments of the invention. In this embodiment, the phototherapy unit 50 includes a light source 200 which supported by a shaft (not shown) and encompassed by a balloon 64. The light source 200 preferably produces white light of sufficient intensity to ablate renal nerves and ganglia of innervated target tissue.

In some embodiments, the light source 200 includes a flash lamp containing a suitable excitable gas, such as xenon or krypton. The light source 200 includes a pair of electrodes 201, 203 situated in a closed transparent vessel that contains the excitable gas. Each electrode 201, 203 is connected to an electrical conductor (e.g., filament) that extends along a length of a catheter to which the phototherapy unit 50 is coupled. The electrical conductors are preferably situated in individual lumens of the catheter with electrical insulation appropriate for the high voltage filaments. An external power source is coupled to the filaments, and controlled to delivery power to the light source 200, causing arcing across the electrode gap and generation of a high intensity flash of white light. According to various low voltage embodiments, the white light source 200 may include one or more solid state light emitting devices (e.g., LEDs) that produce high intensity light having multiple wavelengths.

The balloon 64 incorporates a reflector arrangement 202 that serves to focus light emitted by the light source 200, which is often omni-directional, in a preferred direction. The reflector arrangement 202 may have a parabolic or other desired profile, and the light source 200 may be situated off-axis to the balloon's shaft (not shown for purposes of clarity) and biased toward the reflector arrangement 202. The reflector arrangement 202 may be a coating of aluminum, silver, gold or other reflective material. The profile of the reflector arrangement 202 may be defined by balloon material that is shaped to define the reflector arrangement 202. The profile of the reflector arrangement 202 may be defined by separate material or a pre-fabricated insert that is welded or otherwise attached to the balloon's inner wall to define the shape of the reflector arrangement 202.

The balloon 64 is preferably filled with a cooling fluid which is transparent to the wavelengths of light produced by the light source 200. It is preferred that the balloon 64 be positioned against the artery wall to provide good thermal and optical coupling between the cooling balloon 64 and light source 200, respectively.

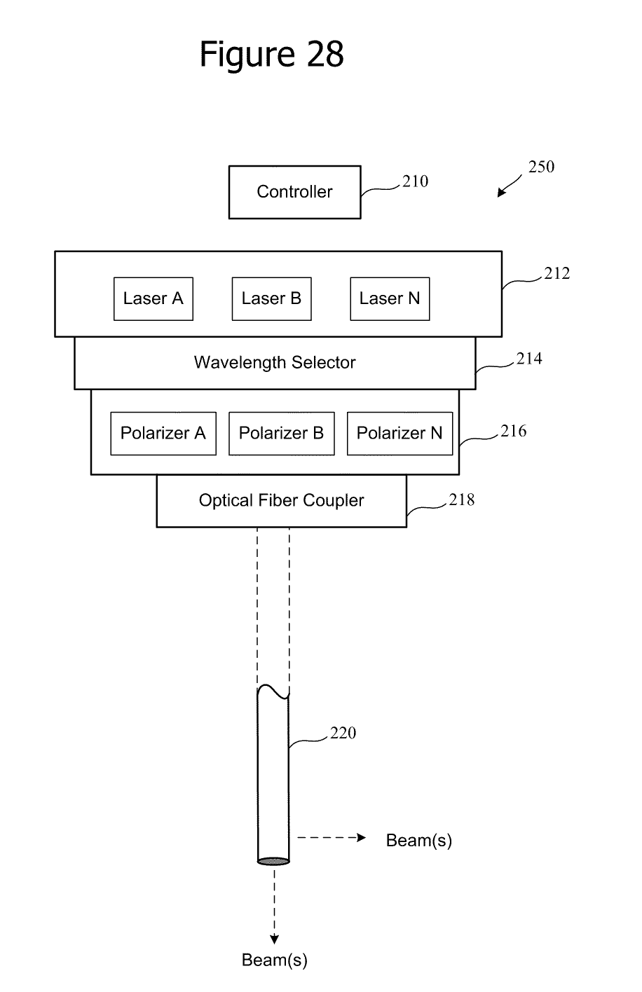

FIG. 28 is a diagram of various laser light source components of a phototherapy system 250 in accordance with embodiments of the invention. The components of the phototherapy system 250 shown in FIG. 28 include a laser light source 212 which can include a number of different laser sources, Laser A, Laser B, and Laser N, for example. The lasers preferably produce laser light of disparate wavelength. For example, Laser A may produce laser light in the green spectrum (e.g., 495-570 nm), Laser B may produce laser light in the near-infrared spectrum (e.g., 700 nm-1400 nm), and Laser N may produce laser light having a wavelength between those associated with Lasers A and B (e.g., 570-700 nm).

A wavelength selector 214 may be included to adjust the wavelength of laser light exiting the phototherapy system 250. The wavelength selector 214 may include one or more gradient-index (GRIN) lenses, for example. The phototherapy system 250 may include one or more polarizers 216 (e.g., Polarizer A, B, N) to polarize the laser source to have a desired polarization (e.g., circular, linear, elliptical, etc.). An optical fiber coupler 218 is used to provide physical and optical coupling between the phototherapy system 250 and a catheter or probe 220 that includes one or more optical fibers. The phototherapy system 250 preferably includes a controller 210 that controls the operation and functions of various system components. It is noted that some of the components shown in FIG. 28 may be optional.

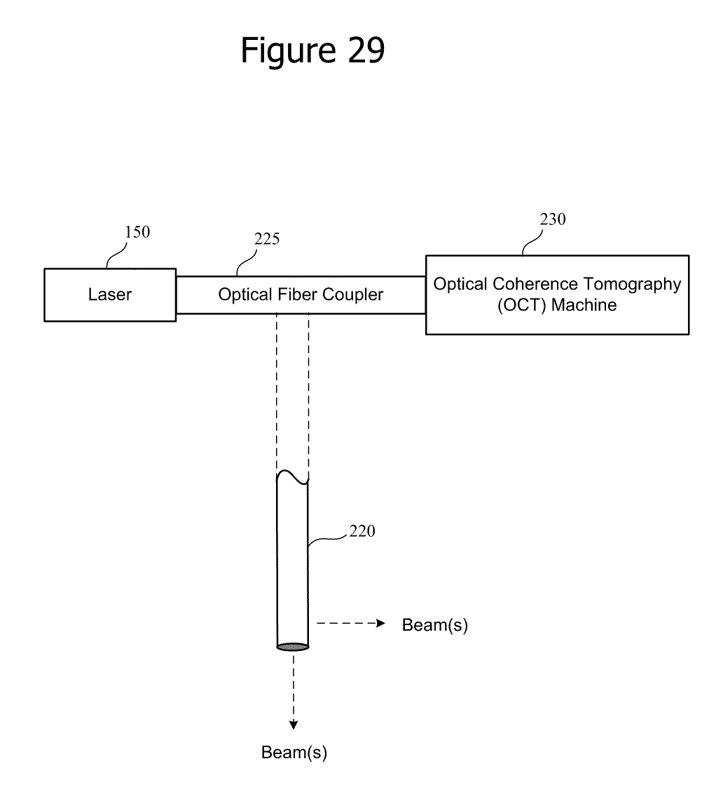

FIG. 29 is a diagram of a system that includes an optical imaging system, an optical ablation system, and an optical coupling arrangement that facilitates quick and easy coupling and decoupling of a catheter or probe to and from the two systems. In the embodiment shown in FIG. 29, a laser 150 is configured for thermally or photoacoustically ablating innervated tissue in a manner previously described. An optical coherence tomography machine 230 is used for obtaining high resolution images of innervated vasculature using low power optical energy. OCT is based on principles of low coherence interferometry. An optical fiber coupler 225 is used to couple and decouple the laser 150 and OCT machine 230 to and from a catheter 220 which includes one or more optical fibers.

In the OCT machine 230, light is broken into a sample arm, which contains the item of interest, and a reference arm, which is typically a mirror. The combination of reflected light from the sample arm and reference light from the reference arm gives rise to an interference pattern, but only if light from both arms have traveled the same optical distance. By scanning the mirror in the reference arm, a reflectivity profile of the sample can be obtained. This reflectivity profile, termed an A-scan, contains information about the spatial dimensions and location of structures within the item of interest. A cross-sectional tomography, termed a B-scan, can be obtained by laterally combining a series of these axial depth scans (A-scans). C-scan imaging at an acquired depth may also be achieved using the OCT machine 230 depending on the imaging engine used.

According to one approach, a catheter 220 is positioned at an intravascular or extravascular location relative to a patient's renal artery. Because the signal-to-noise ratio drops off considerably at imaging depths greater than about 1 mm using conventional OCT, an extravascular location may be most efficacious. Access to extravascular locations of the renal artery may be achieved using intra-to-extra vascular access via the renal vein or inferior vena cava. The distal end of the catheter 220 is moved relative to the outer wall of the renal artery for locating the renal nerve and renal/aortal ganglia using the OCT machine 230.

After a renal nerve or ganglion is located using the OCT machine 230, the proximal end of the catheter 220 is decoupled from the optical fiber coupler 225 while the distal end is maintained at its current location (i.e., above the target renal nerve or ganglion). The laser 150 is then coupled to the proximal end of the catheter 220 via the optical fiber coupler 225. Light generated by the laser 150 is transmitted from the laser and along the catheter 220 to the target tissue for ablating the renal nerve or ganglion included in the target tissue.

The proximal end of the catheter 220 may then be decoupled from the laser 150 and coupled to the OCT machine 230, and scanning of the renal artery or abdominal aorta may be continued to locate another target renal nerve or ganglion. This method may be repeated until renal derivation is completed. It is noted that coupling and decoupling using optical fiber coupler 225 may involve manual coupling/decoupling effort or optical switching that obviates manual coupling/decoupling.

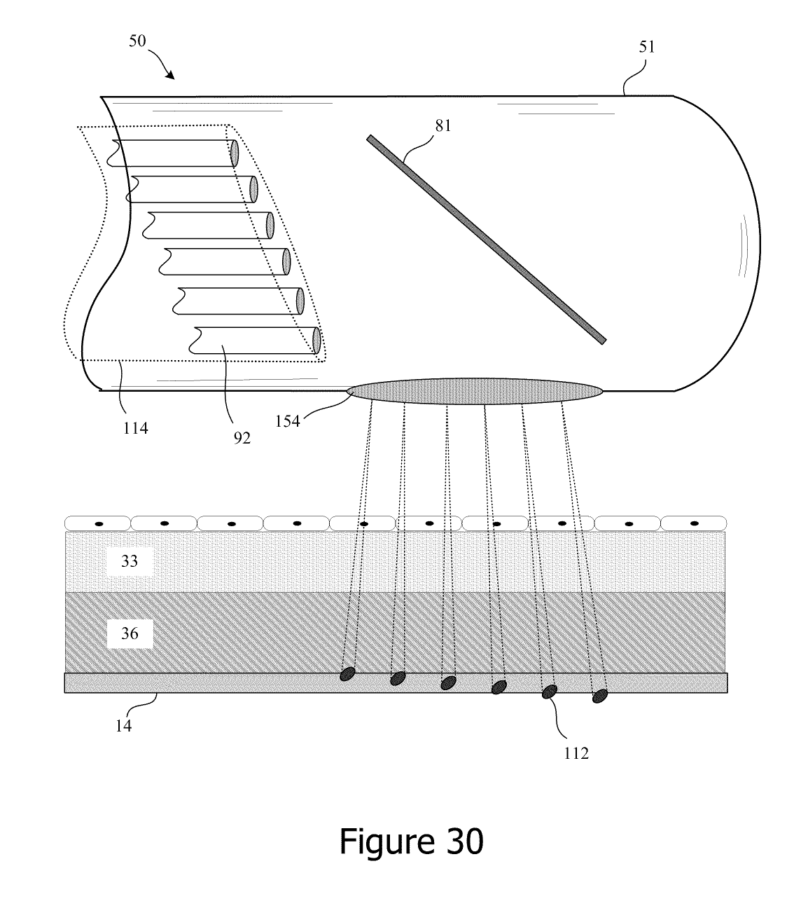

FIG. 30 illustrates an embodiment of a phototherapy unit 50 in accordance with embodiments of the invention. In FIG. 30, a multiplicity of optical fibers 92 of a fiber bundle 114 are arranged in an offset configuration at the distal end of a catheter 51. A manifold may be used to support the optical fibers 92 and maintain them in the desired offset configuration. Laser light emitted from the array of offset optical fibers 92 is directed by a mirror or prism 81 through an optics arrangement 154 and into tissue of a renal artery wall. The optics arrangement 154 in this embodiment may include an objective lens that is used to displace focused spots 112 in the longitudinal and transverse dimensions at a desired depth in the renal artery wall tissue 36.

The spots 112 may be scanned to create a multidimensional image when operating the phototherapy unit 50 in an imaging mode. Ablation spots 112 may be produced by the phototherapy unit 50 to create a multidimensional lesion when operating the phototherapy unit 50 in a phototherapy delivery mode. As shown in FIG. 30, the spots 112 are formed at a depth consistent with a depth of the renal nerves 14 and/or ganglia of the renal artery. As is further shown in FIG. 30, the spots may be staggered in terms of depth to improve the imaging and cutting of renal nerves 14.

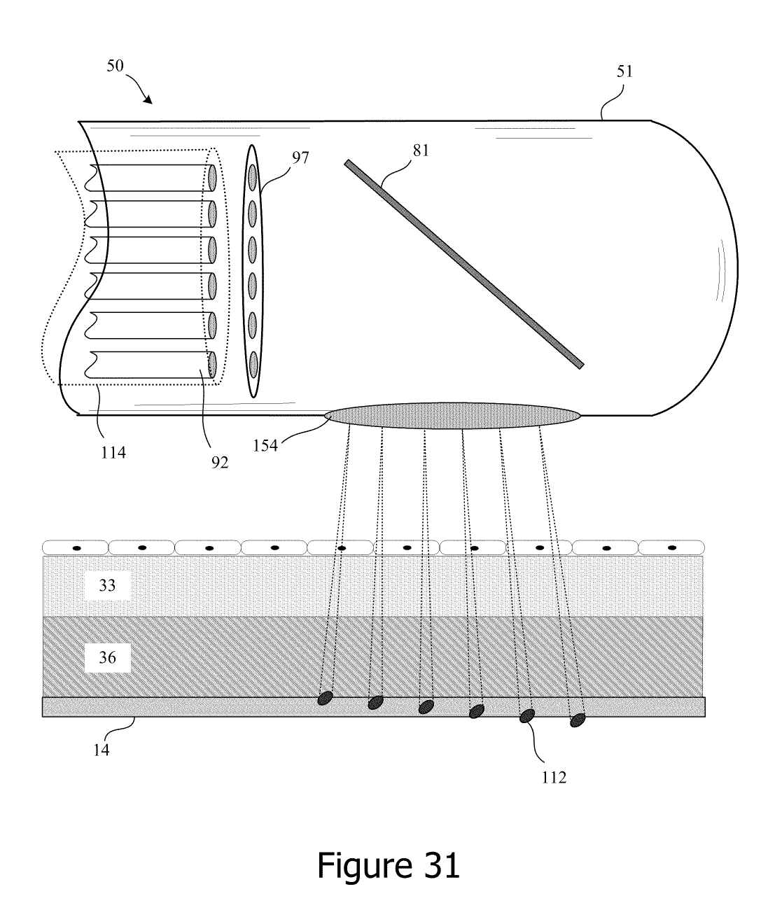

In another embodiment, as is shown in FIG. 31, a multiplicity of mirrors 81 may be arranged to define a diffraction grating which displaces focused spots 112 in the longitudinal and transverse dimensions at a desired depth in the renal artery wall tissue 36. In this embodiment, laser light emitted from an array of aligned optical fibers 92 passes through a miocrolens array 97 and is directed by the diffraction grating 81 through an optics arrangement 154 and into tissue of a renal artery wall. The optics arrangement 154 in this embodiment may include an objective lens that is used to displace focused spots 112 in the longitudinal and transverse dimensions at a desired depth in the renal artery wall tissue 36.

The spots 112 may be scanned to create a multidimensional image when operating the phototherapy unit 50 in an imaging mode. When operating the phototherapy unit 50 in an phototherapy delivery mode, ablation spots 112 may be produced to create a multidimensional lesion in the artery wall tissue. The spots 112 are preferably formed at a depth consistent with a depth of the renal nerves 14 and/or ganglia of the renal artery, and may be staggered in terms of depth to improve the imaging and cutting of renal nerves 14.

It is noted that a phototherapy unit 50 in accordance with various embodiments of the invention may incorporate multiple phototherapy units 50 for purposes of imaging and/or ablating target tissue. Some of the phototherapy units 50, for example, can operate continuously in a scan mode, while other phototherapy units 50 can be operated continuously, intermittently, or sequentially in an ablation mode.

It is further noted that marker bands can be placed on one or multiple parts of the catheter 51 to enable visualization during the delivery, imaging, and/or denervation procedures. The marker bands may be solid or split bands of platinum or other radiopaque metal, for example.

Although generally described in the context of renal artery deployment herein, embodiments of the invention may be configured for deployment from the renal vein or inferior vena cava and advanced to an extravascular location proximate the renal artery. An advantage to using a phototherapy unit 52 deployed from the renal vein or inferior vena cava concerns a reduced risk of injuring the intima and other tissues of the renal artery, since extravascular phototherapy delivery need only involve the outer adventitial tissue of the renal artery.

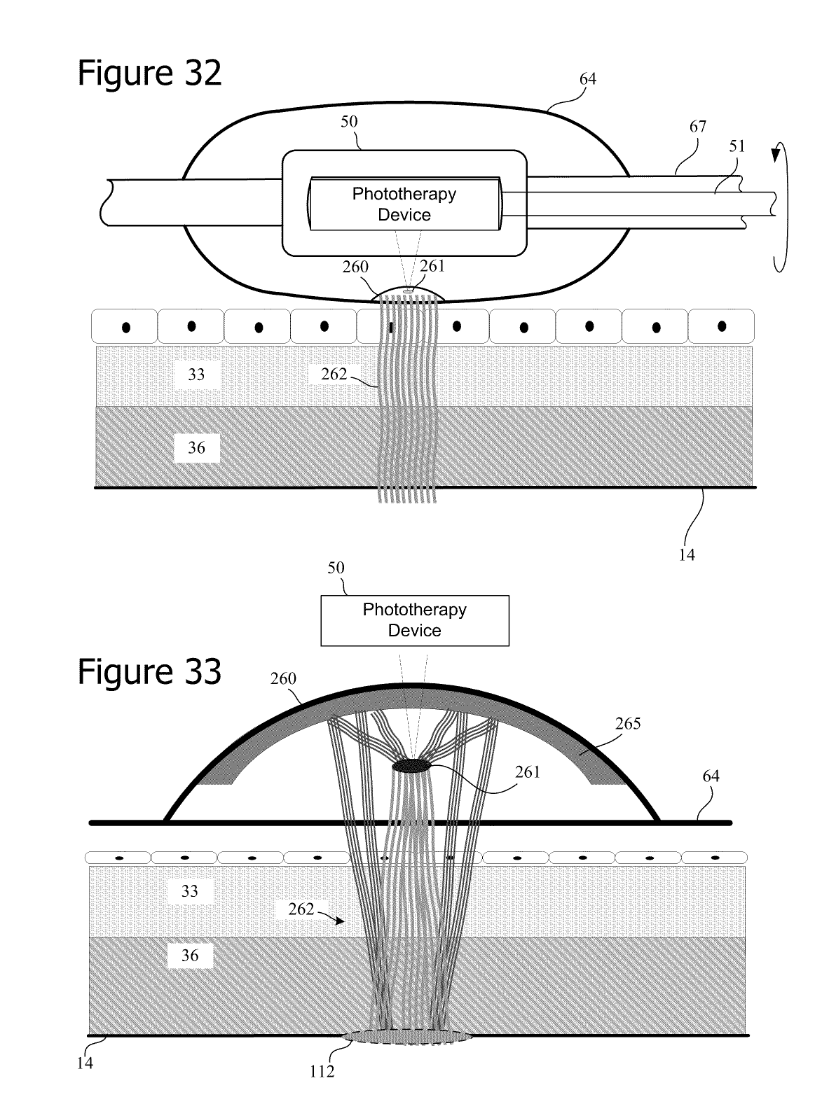

FIGS. 32 and 33 illustrate an apparatus for denervating a patient's renal artery using a photoacoustic ablation arrangement in accordance with embodiments of the invention. In FIG. 32, a phototherapy unit 50, supported by a catheter 51, is situated within a balloon 64. The balloon 64 incorporates a fluid pouch, bladder or channel (referred to as fluid vessel 260) disposed on a peripheral portion of the balloon 64. The fluid vessel 260 may be extend along all or a radial section of the circumference of the balloon 64. In one embodiments, the fluid vessel 64 defines a channel that is fluidly coupled to a circulating coolant path via a lumen arrangement provided within a shaft 67 of the balloon 64. In other embodiments, the fluid vessel 64 may be configured as an isolated pouch containing a water based fluid or gel.

In FIGS. 32 and 33, laser and optics arrangements of the phototherapy unit 50 are configured to deliver optical energy to the fluid contained within the fluid vessel 261. In embodiments where coolant circulation is used, coolant movement within the fluid vessel 64 may be temporarily halted during delivery of optical energy to the fluid vessel 64. Laser light emitted by the phototherapy unit 50 is directed to a focal line, area or volume (or multiple foci) within the fluid vessel 261 which creates a cavitation bubble 261 therein. Light is emitted from the phototherapy unit 50 sufficient to cause the bubble 261 to burst, thereby generating an acoustic shock wave which is directed to target tissue 112 that includes a renal nerve 14 or ganglion.

The fluid vessel 261 may incorporate an acoustic reflector 265 to aid in focusing the acoustic shock wave to the target tissue 112, as best seen in FIG. 33. Propagation of the acoustic shock wave through the innervated target tissue 112 fractures neural sheaths of nerve fibers of the target renal nerve 14 or ganglion, preferably causing permanent nerve cell disruption sufficient to prevent nerve regeneration. The phototherapy unit 50 may be configured for rotation within the balloon 64 in response to movement of a manual or motorized rotation mechanism coupled to the catheter 51 or the phototherapy unit itself. By rotating the phototherapy unit 50 and creating a series of cavitation bubble implosions or explosions, a circular lesion can be created in the outer adventitia 36 and/or vasa vasorum that cuts renal nerves and ganglion and permanently terminates all renal sympathetic nerve activity.

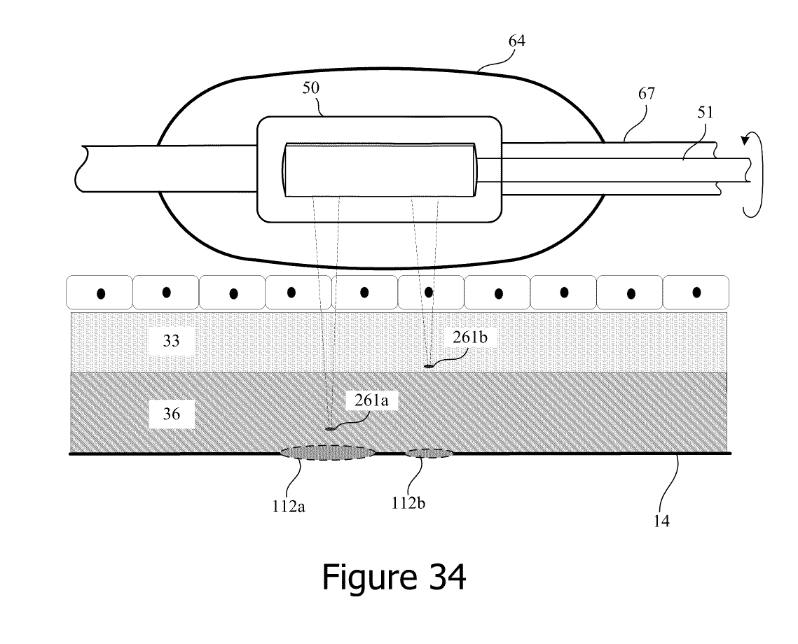

FIG. 34 illustrates another apparatus for denervating a patient's renal artery using a photoacoustic ablation arrangement in accordance with embodiments of the invention. In FIG. 34, a phototherapy unit 50 is supported by a catheter 51 and situated within a balloon 64. In this embodiment, laser and optics arrangements of the phototherapy unit 50 are configured to deliver optical energy to a focal line, area or volume (or multiple foci) within the media 33, adventitia 36, or vasa vasorum.

Optical energy deposited at the focal line, area or volume creates a cavitation bubble 261a in the target tissue. For illustrative purposes, FIG. 34 shows formation of cavitation bubbles 261a and 261b in the media 33 and adventitia 36, respectively. Light is emitted from the phototherapy unit 50 sufficient to cause the bubbles 261a, 261b to burst, thereby generating acoustic shock waves which are directed to target tissue 112a and 112b that includes a renal nerve 14 or ganglion. It is noted that forming a cavitation bubble 261a relatively close to the target tissue 112a may result in creation of a larger lesion 112a when compared to a cavitation bubble 261b formed relatively far from the target tissue 112b.

In some embodiments, the light emitted from the phototherapy unit 50 can be controlled to grow and/or launch a cavitation bubble in the direction of target tissue 112. For example, and with reference to FIG. 34, a cavitation bubble 261b can be formed in the media 33. Optical energy can be controllably deposited to cause the cavitation bubble 261b to grow volumetrically, which results in a reduction in spacing between the growing bubble 261b and the target tissue 112b. Growth of the bubble 261b can be monitored using imaging provided by the phototherapy unit 50 or a separate internal or external imaging device or system. Upon reaching a desired size or location, additional optical energy can be deposited sufficient to cause the bubble 261b to burst, creating a relatively large lesion in the target tissue 112b.

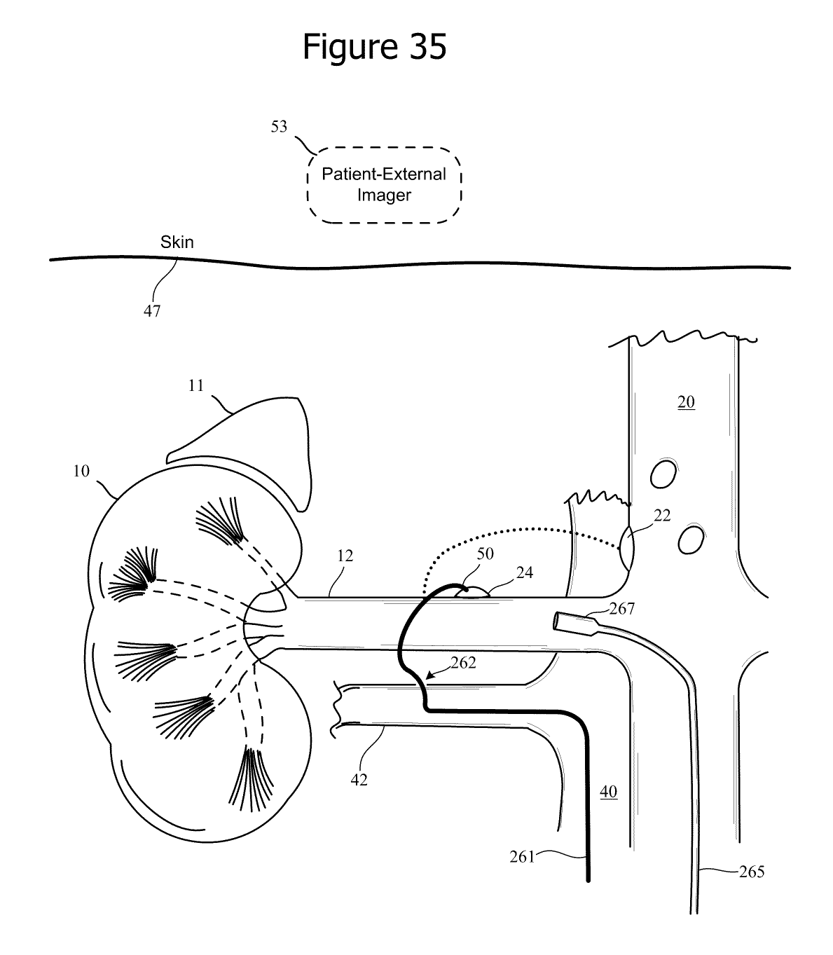

FIG. 35 illustrates an apparatus for facilitating guided delivery of a phototherapy catheter to innervated tissue and ganglia that contribute to renal sympathetic nerve activity in accordance with embodiments of the invention. According to various embodiments, a phototherapy catheter 261 is used cooperatively with an imaging system to locate target renal nerves and ganglia to be ablated using optical energy. In FIG. 35, the phototherapy catheter 261 is configured for intra-to-extra vascular deployment, and the imaging system may include an intravascular imaging catheter 265 or an external imager 53 of a type previously described.

According to some embodiments, an intravascular imaging catheter 265 is delivered to a location within a patient's renal artery 12, typically accessed via the inferior abdominal aorta 20. The intravascular imaging catheter 265 preferably includes an imaging device 267, such as an IVUS device or other ultrasonic imaging device, or a laser imaging device, such as a laser transducer or other optical imaging device. With the imaging device 267 properly positioned in or proximate the renal artery 12, the phototherapy catheter 261 is advanced into the renal vein 42, typically accessed via the inferior vena cava 40. The phototherapy catheter 261 preferably includes a steering mechanism. Suitable steering mechanisms that can be incorporated in a phototherapy catheter 261 of the present invention include various mechanisms incorporated into known steerable guide catheters.

The phototherapy catheter 261 includes an optical arrangement of a type previously described. Using the phototherapy catheter 261 positioned adjacent a renal vein wall location, optical energy is deposited to create an access hole 262 in the renal vein 42. With aid from the imaging catheter 265 or external imager 53, the phototherapy catheter 261 is advanced through the access hole 262 and navigated around the exterior of the renal artery 12 to a location adjacent a target nerve or ganglion, such as a renal ganglion 24 as shown in FIG. 35.

Optical energy is deposited using the phototherapy catheter 261 to ablate the target tissue in a manner previously described, so that all renal sympathetic nerve activity associated with nerve fibers included within the target tissue is permanently terminated. The phototherapy catheter 261 can be navigated to another location of the renal artery or abdominal aorta 20, such as a location of the renal artery 12 that includes a renal nerve, the aorticorenal ganglion 22, the superior mesenteric ganglion, or the celiac ganglia or plexus. The imaging catheter 267 is preferably moved to an appropriate intravascular location to aid navigation and positioning of the phototherapy catheter 261, such as a location within the abdominal aorta 20 or renal vein 40.

In accordance with various embodiments described herein, one or more physiologic parameters can be monitored during the ablation procedure to determine the effect of the ablation on the patient's renal sympathetic nerve activity. For example, an electrode arrangement may be situated in contact with the inner or outer wall of the renal artery 12 near opposing sides of the renal artery 12. The electrode arrangement may be configured to measure nerve impulses transmitted along renal nerve fibers. By way of further example, one or more physiological parameters that are sensitive to changes in renal sympathetic nerve activity may be monitored, and the efficacy of the ablation procedure may be determined based on measured changes in the physiological parameter(s). Suitable apparatuses for these purposes are disclosed in commonly owned U.S. Patent Publication No. 2008/0234780 and in U.S. Pat. No. 6,978,174, which are incorporated herein by reference.

It is noted that marker bands can be placed on one or multiple parts of the phototherapy catheter 261 and/or imaging catheter 261 to enable visualization during the delivery, imaging, and/or denervation procedures. The marker bands may be solid or split bands of platinum or other radiopaque metal, for example.

Referring now to FIG. 36, a catheter 51 to which a phototherapy unit 50 of the present invention is connected may incorporate a hinge mechanism 356 built into the catheter 51 proximate the phototherapy unit 50. The hinge mechanism 356 may be built into other elongated intravascular device embodiments of the disclosure, such as shaft 67 and shaft 88 of balloons 64 shown in several of the figures of the disclosure. The hinge mechanism 356 is constructed to enhance user manipulation of the catheter 51 when navigating around a nearly 90 degree turn from the abdominal aorta into the renal artery. It is understood that one or more hinge mechanisms 356 may be built into other catheters and sheaths that may be used to facilitate access to the renal artery via the abdominal aorta. For example, a delivery sheath or guide catheter that is used to provide renal artery access for a catheter 51 of a type described herein may incorporate one or more hinge mechanisms 356.

FIG. 36 illustrates a portion of the catheter 51 that incorporates a hinge mechanism 356 in accordance with embodiments of the invention. The hinge mechanism 356 is provided at a location of the catheter 51 between a proximal section 352 and a distal section 354 of the catheter's shaft. The hinge mechanism 356 is preferably situated near the proximal section of the phototherapy unit 50. According to various embodiments, the hinge mechanism 356 comprises a slotted tube arrangement that is configured to provide a flexible hinge point of the catheter's shaft proximate the phototherapy unit 50.

The catheter's shaft may be formed to include an elongate core member 357 and a tubular member 353 disposed about a portion of the core member 357. The tubular member 353 may have a plurality of slots 361 formed therein. The slotted hinge region 356 of the catheter's shaft may be configured to have a preferential bending direction.

For example, the tubular member 352 may have a plurality of slots 361 that are formed by making a pair of cuts into the wall of tubular member 361 that originate from opposite sides of tubular member 353, producing a lattice region of greater flexibility relative to the proximal and distal sections 352, 354 of the catheter's shaft. The thickness of the catheter wall at the hinge region 356 can be varied so that one side of the catheter wall is thicker than the opposite side. This difference in wall thickness alone or in combination with a difference in slot (void) density at the hinge region 356 provides for a preferential bending direction of the distal portion of the catheter 51.

A hinge arrangement 356 constructed to provide for a preferential bending direction allows a physician to more easily and safely navigate the phototherapy unit 50 to make the near 90 degree turn into the renal artery from the abdominal aorta, for example. One or more marker bands may be incorporated at the hinge region 356 to provide visualization of this region of the catheter's shaft during deployment. Details of useful hinge arrangements that can be incorporated into embodiments of a catheter 51 of the present invention or other component that facilitates access to the renal artery/vein from the abdominal aorta are disclosed in U.S. Patent Publication Nos. 2008/0021408 and 2009/0043372, which are incorporated herein by reference. It is noted that the catheter 51 may incorporate a steering mechanism in addition to, or exclusion of, a hinge arrangement 356. Known steering mechanisms incorporated into steerable guide catheters may be incorporated in various embodiments of a catheter 51 of the present invention.

The discussion provided herein concerning degrees of induced renal nerve damage, temperature ranges, amount of energy or power delivered into target tissue, and other embodiment details described above are provided for non-limiting illustrative purposes. Actual therapeutic parameters associated with the denervation apparatuses and methodologies may vary somewhat or significantly from those described herein, and be impacted by a number of factors, including patient-specific factors (e.g., the patient's unique renal vasculature and sympathetic nervous system characteristics), refractoriness to drugs impacting renal function, type and technology of the therapy device(s), therapy duration and frequency, use of a single therapy device or multiplicity of therapy devices (in sequential or concurrent use), structural characteristics of the therapy device(s) employed, and other implementation and physiologic particulars, among others.

The foregoing description of the various embodiments of the invention has been presented for the purposes of illustration and description. It is not intended to be exhaustive or to limit the invention to the precise form disclosed. Many modifications and variations are possible in light of the above teaching. For example, the devices and techniques disclosed herein may be employed in vasculature of the body other than renal vasculature, such as coronary and peripheral vessels and structures. By way of further example, embodiments of a phototherapy unit may be implemented for chronic use, and structures other than a catheter, such as a stent, may be used to maintain positioning of the phototherapy unit within the renal artery or other vessel. It is intended that the scope of the invention be limited not by this detailed description, but rather by the claims appended hereto.

What is claimed is:

1. An apparatus for facilitating delivery of optical energy to a renal artery of a patient, comprising:
a catheter configured for deployment relative to the renal artery;
an optical fiber arrangement supported by the catheter and comprising a coupling for receiving laser light from a laser light source; and
an optics arrangement supported by the catheter and coupled to the optical fiber arrangement, the optics arrangement comprising one or more optical elements arranged to receive the laser light and project optical energy to a desired depth within innervated tissue at or proximate an outer wall of the renal artery, the optical energy of sufficient power to ablate innervated tissue at or proximate the outer wall of the renal artery.

2. The apparatus according to claim 1, wherein the optical energy is sufficient to ablate the innervated renal artery tissue with negligible injury to inner wall tissue of the renal artery.

3. The apparatus according to claim 1, wherein the one or more optical elements are configured to project the optical energy from the catheter to the innervated renal artery tissue in a circular pattern, the optical energy sufficient to ablate the innervated renal artery tissue with negligible injury to inner wall tissue of the renal artery.

4. The apparatus according to claim 1, wherein the one or more optical elements are configured to project the optical energy from the catheter to the innervated renal artery tissue in a spiral, the optical energy sufficient to ablate the innervated renal artery tissue with negligible injury to inner wall tissue of the renal artery.

5. The apparatus according to claim 1, wherein:
in a first mode of operation, the optics arrangement is configured to project optical energy to innervated tissue at or proximate the outer wall of the renal artery for scanning the innervated tissue; and
in a second mode of operation, the optics arrangement is configured to project optical energy to innervated tissue at or proximate the outer wall of the renal artery for ablating the innervated tissue.

6. The apparatus according to claim 1, wherein the one or more optical elements are configured to convert a parallel light beam received from the optical fiber arrangement into an image having a predetermined shape and project the image to a desired depth within the innervated tissue of the renal artery.

7. The apparatus according to claim 1, wherein the laser light source comprises a continuous wave laser, and the optics arrangement is configured to direct optical energy to innervated tissue of the renal artery for effecting thermal ablation of the innervated tissue.

8. The apparatus according to claim 1, wherein the laser light source comprises an ultrafast laser, and the optics arrangement is configured to direct optical energy to innervated tissue of the renal artery for effecting non-thermal ablation of the innervated tissue.

9. The apparatus according to claim 1, wherein the optical energy is sufficient to create a cavitation bubble in the innervated renal artery tissue at a predetermined depth, the cavitation bubble creating a rupture in the innervated renal artery tissue upon bursting.

10. The apparatus according to claim 1, wherein the optics arrangement is configured to redirect light propagated along the optical fiber arrangement through a surface of the catheter that extends along all or a portion of a circumference of the catheter.

11. The apparatus according to claim 1, wherein:
the optics arrangement comprises a mirror and at least one lens, the mirror redirecting light propagated along the optical fiber arrangement through the at least one lens and out of the catheter; and
the mirror is configured for rotation within the catheter in response to movement of a manual or motorized rotation mechanism coupled to the mirror.

12. The apparatus according to claim 1, further comprising a balloon arrangement dimensioned for deployment within a lumen of the renal artery, the balloon arrangement supporting at least a portion of the optical fiber arrangement and the optics arrangement at a relatively fixed location within the renal artery lumen when the balloon arrangement is expanded in its deployed configuration.

13. The apparatus according to claim 12, wherein the balloon arrangement is configured to receive a thermal transfer fluid.

14. The apparatus according to claim 1, wherein:
the laser light source comprises a plurality of lasers configured to produce light having a plurality of disparate wavelengths; and
the optics arrangement comprises a plurality of optical elements arranged to direct the light from the plurality of lasers to innervated tissue of the renal artery from disparate angles.

15. The apparatus according to claim 1, wherein the optics arrangement comprises an axicon lens, a cylindrical lens, or a toroidal lens.

16. The apparatus according to claim 1, wherein the catheter is configured for intravascular deployment relative to the renal artery.

17. The apparatus according to claim 1, wherein the catheter is configured for extravascular deployment relative to the renal artery.

18. The apparatus according to claim 1, comprising an optical coherence tomography (OCT) machine and an optical fiber coupler, wherein:
in a first mode, the OCT machine is coupled to the catheter via the optical fiber coupler for imaging the innervated tissue and locating target tissue of the innervated tissue; and
in a second mode, the laser light source is coupled to the catheter via the optical fiber coupler for ablating the target tissue located by the OCT machine.

19. An apparatus for facilitating delivery of optical energy to a renal artery of a patient, comprising:
a catheter configured for intravascular deployment within a lumen of the renal artery;
an optical fiber arrangement supported by the catheter and comprising a coupling for receiving laser light from a laser light source;
an optics arrangement supported by the catheter and coupled to the optical fiber arrangement, the optics arrangement comprising one or more optical elements arranged to receive the laser light; and
a balloon arrangement dimensioned for deployment within the lumen of the renal artery and comprising a fluid vessel containing at least water, the balloon arrangement encompassing at least a portion of the optical fiber arrangement and the optics arrangement, the optical fiber and optics arrangements configured to direct optical energy to the fluid vessel sufficient to create a cavitation bubble therein, the fluid vessel serving to direct an acoustic shock wave generated by bursting of the cavitation bubble to innervated target tissue of the renal artery.

20. An apparatus for facilitating delivery of optical energy to a renal artery of a patient, comprising:
a catheter configured for intravascular deployment within a lumen of the renal artery;
a phototherapy unit provided at a distal end of the catheter, the phototherapy unit comprising a light source configured to generate white light of an intensity sufficient to ablate innervated tissue of the renal artery; and
a balloon arrangement dimensioned for deployment within the lumen of the renal artery and encompassing at least a portion of the phototherapy unit that comprises the white light source, the balloon comprising:
a reflector arrangement disposed on a region of the balloon proximate the phototherapy unit, the reflector arrangement serving to direct white light generated by the light source to innervated target tissue of the renal artery; and
a thermal transfer arrangement configured to provide cooling to renal artery tissue adjacent the balloon.
